SEQUENCE LISTING (2 sequences)

SEQ ID NO: 1 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 2 - protein, 238 aa, Artificial Sequence
  Synthetic
MSKGEELFTGVVPILVELDGDVNGHKFSVSGEGEGDATCGKLTLKFICTTGKLPVPWPTLVTTLSYGMQCFSRYPDHMKQHDFFKSAMPEGYVQERTIFFKDDGNYKTRAEVKFEGDTLVNRIELKGIGFKEDGNILGHKLEYNYNSHNVYIMADKQKNGIKANFRIRHNIEDGSVQLADHYQQNTPIGDVPVLLPDNHYLSTQSALSKDPNEKRDHMVLLEFVTAAGITHGMDELYK

(19) United States
(12) Patent Application Publication
(10) Pub. No.: US 2022/0270711 A1
(43) Pub. Date: Aug. 25, 2022

(54) MACHINE LEARNING GUIDED POLYPEPTIDE DESIGN

(71) Applicant: Flagship Pioneering Innovations VI, LLC, Cambridge, MA (US)

(72) Inventors: Jacob D. Feala, Franklin, MA (US); Andrew Lane Beam, Jamaica Plain, MA (US); Molly Krisann Gibson, Medford, MA (US); Bernard Joseph Cabral, Boston, MA (US)

(21) Appl. No.: 17/597,844

(22) PCT Filed: Jul. 31, 2020
(86) PCT No.: PCT/US2020/044646

§ 371 Date: Jan. 26, 2022

Related U.S. Application Data
(60) Provisional application No. 62/882,150, filed on Aug. 2, 2019.
(60) Provisional application No. 62/882,159, filed on Aug. 2, 2019.

(51) Int. Cl. G16B 40/30; G16B 15/20; G16B 45/00; G16B 35/10

(52) U.S. Cl. CPC G16B 40/30; G16B 35/10; G16B 45/00; G16B 15/20

(57) ABSTRACT

Systems, apparatuses, software, and methods for engineering amino acid sequences configured to have specific protein functions or properties. Machine learning is implemented by methods to process an input seed sequence and generate as output an optimized sequence having the desired function or property.

### RELATED APPLICATIONS

This application claims the benefit of U.S. Provisional Application Nos. 62/882,150 and 62/882,159 both filed on Aug. 2, 2019. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: GBD_SeqListing_ST25.txt; created Jul. 29, 2020, 5 KB in size.

### BACKGROUND

Proteins are macromolecules that are essential to living organisms and carry out or are associated with multitudes of functions within organisms, including, for example, catalyzing metabolic reactions, facilitating DNA replication, responding to stimuli, providing structure to cells and tissue, and transporting molecules. Proteins are made of one or more chains of amino acids and typically form three dimensional conformations.

### SUMMARY

Described herein are systems, apparatuses, software, and methods for generating or modifying protein or polypeptide sequences to achieve a function and/or property, or improvement thereof. The sequences can be determined in silico through computational methods. Artificial intelligence or machine learning is utilized to provide a novel framework for rationally engineering proteins or polypeptides. Accordingly, new polypeptide sequences distinct from naturally occurring proteins can be generated to have a desired function or property.

Design of amino acid sequences (e.g., proteins) for a specific function has long been a goal of molecular biology. However, protein amino acid sequence prediction based on a function or property is highly challenging due at least in part to the structural complexity that can arise from what is seemingly a simple primary amino acid sequence. One approach to date has been the use of in vitro random mutagenesis followed by selection, resulting in a directed evolution process. However, such approaches are time and resource-intensive, typically requiring generation of mutant clones, such generation, in turn, subject to biases in library design or limited exploration of sequence space, screening those clones for the desired properties, and iteratively repeating this process. Indeed, the traditional approach has failed to provide an accurate and reproducible method for predicting protein function based on an amino acid sequence, much less allow for predicting an amino acid sequence based on a protein function. In fact, traditional thinking with respect to protein primary sequence prediction based on function is that a primary protein sequence cannot be directly associated with a known function, because so much of the proteins function is driven by its ultimate tertiary (or quaternary) structure.

By contrast, having the ability to engineer proteins having a property or function of interest using computational or in silico methods could transform the field of protein design. Despite much study on the subject, little success has been achieved thus far. Accordingly, disclosed herein are innovative systems, apparatuses, software, and methods that generate an amino acid sequence coding for a polypeptide or protein configured to have a particular property and/or function. Therefore, the innovations described herein are unexpected and produce unexpected results in view of traditional thinking with respect to protein analysis and protein structure.

Described herein is a method of engineering an improved biopolymer sequence as assessed by a function, comprising: (a) calculating a change in the function with regard to an embedding at a starting point according to a step size, the starting point provided to a system comprising a supervised model that predicts the function of a biopolymer sequence and a decoder network, the supervised model network comprising an encoder network providing the embedding of biopolymer sequences in a functional space representing the function and the decoder network trained to provide a probabilistic biopolymer sequence, given an embedding of a biopolymer sequence in the functional space, optionally wherein the starting point is the embedding of a seed biopolymer sequence, thereby providing a first updated point in the functional space; (b) optionally calculating a change in the function with regard to the embedding at the first updated point in the functional space and optionally iterating the process of calculating a change in the function with regard to the embedding at a further updated point; (c) upon approaching a desired level of the function at the first updated point in the functional space, or optionally iterated further updated point, providing the first updated point, or optionally iterated further updated point to the decoder network; and (d) obtaining a probabilistic improved biopolymer sequence from the decoder.

Herein, a double meaning may be associated with the term “function”. On the one hand, the function may represent, in a qualitative aspect, some property and/or capability (like, for example, fluorescence) of the protein in the biological domain. On the other hand, the function may represent, in a quantitative aspect, some figure of merit associated with that property and/or capability in the biological domain, e.g., a measure for the strength of a fluorescent effect.

Therefore, the meaning of the term “functional space” is not limited to its meaning in the mathematical domain, namely a set of functions that all take in an input from one and the same space and map this input to an output in the same or other space. Rather, the functional space may comprise compressed representations of biopolymer sequences from which the value of the function, i.e. the quantitative figure of merit for the desired property and/or capability, may be obtained.

In particular, the compressed representations may comprise two or more numeric values that may be interpreted as coordinates in a Cartesian vector space having two or more dimensions. However, that Cartesian vector space may not be completely filled with these compressed representations. Rather, the compressed representations may form a sub-space within said Cartesian vector space. This is one meaning of the term “embedding” used herein for the compressed representations.

In some embodiments, the embedding is a continuously differentiable functional space representing the function and having one or more gradients. In some embodiments, calculating the change of the function with regard to the embedding comprises taking a derivative of the function with regard to the embedding.

In particular, the training of the supervised model may tie the embedding to the function in the sense that if two biopolymer sequences have similar values of said figure of merit in the quantitative sense of the function, their compressed representations are close together in the functional space. This facilitates making targeted updates to the compressed representations in order to arrive at a biopolymer sequence that has an improved figure of merit.

The phrase “having one or more gradients” is not to be construed limiting in the sense that this gradient has to be computed on some explicit function mapping a compressed representation to a quantitative figure or merit. Rather, the dependency of that figure of merit on the compressed representation may be a learned relationship for which no explicit functional term is available. For such a learned relationship, gradients in the functional space of the embedding may, for example, be computed by means of backpropagation. For example, if a first compressed representation of a biopolymer sequence in the embedding is transformed into a biopolymer sequence by the decoder, and this biopolymer sequence is in turn fed into the encoder and mapped to a compressed representation, the supervised model may then compute said quantitative figure of merit from this compressed representation. A gradient of this figure of merit with respect to the numerical values in the original compressed representation may then be obtained by means of backpropagation. This is illustrated in FIG. 3A in more detail.

As noted before, a particular embedding space and a particular figure of merit may be two faces of the same medal in that compressed representations with similar figures of merit are close together in the embedding space. Therefore, if there is a meaningful way to obtain a gradient of the figure of merit function with respect to the numeric values that make up the compressed representations, then that embedding space may be considered “differentiable”.

The term “probabilistic biopolymer sequence” may, in particular, comprise some distribution of biopolymer sequences from which a biopolymer sequence may be obtained by sampling. For example, if a biopolymer sequence of a defined length L is sought, and the set of available amino acids for each position is fixed, the probabilistic biopolymer sequence may indicate, for each position in the sequence and each available amino acid, a probability that this position is occupied by this particular amino acid. This is illustrated in FIG. 3C in more detail.

In some embodiments, the function is a composite function of two or more component functions. In some embodiments, the composite function is a weighted sum of the two or more composite functions. In some embodiments, two or more starting points in the embedding are used concurrently, e.g., at least two starting points. In embodiments, 2, 3, 4, 5, 6, 7, 8, 9, 10, 20, 30, 40, 50, 100, 200 starting points can be used concurrently, however this is a non-limiting list. In some embodiments, correlations between residues in a probabilistic sequence comprising a probability distribution of residue identities are considered in a sampling process using conditional probabilities that account for the portion of the sequence that has already been generated. In some embodiments, the method further comprises selecting the maximum likelihood improved biopolymer sequence from a probabilistic biopolymer sequence comprising a probability distribution of residue identities. In some embodiments, the method further comprises sampling the marginal distribution at each residue of a probabilistic biopolymer sequence comprising a probability distribution of residue identities. In some embodiments, the change of the function with regard to the embedding, is calculated by calculating the change of the function with regard to the encoder, then the change of the encoder to the change of the decoder, and the change of the decoder with regard to the embedding. In some embodiments, the method comprises: providing the first updated point in the functional space or further updated point in the functional space to the decoder network to provide an intermediate probabilistic biopolymer sequence, providing the intermediate probabilistic biopolymer sequence to the supervised model network to predict the function of the intermediate probabilistic biopolymer sequence, then calculating the change in the function with regard to the embedding for the intermediate probabilistic biopolymer to provide a further updated point in the functional space.

Described herein is a system comprising a processor; and a non-transitory computer readable medium encoded with software configured to cause the processor to: (a) calculate a change in the function with regard to an embedding at a starting point according to a step size, thereby providing a first updated point in the functional space, the starting point provided to a system comprising a supervised model that predicts the function of a biopolymer sequence and a decoder network, the supervised model network comprising an encoder network providing the embedding of biopolymer sequences in a functional space representing the function and the decoder network trained to provide a probabilistic biopolymer sequence, given an embedding of a biopolymer sequence in the functional space, optionally wherein the starting point is the embedding of a seed biopolymer sequence; (b) optionally calculate a change in the function with regard to the embedding at the first updated point in the functional space and optionally iterating the process of calculating a change in the function with regard to the embedding at a further updated point; (c) upon approaching a desired level of the function at the first updated point in the functional space, or optionally iterated further updated point, provide the first updated point, or optionally iterated further updated point to the decoder network; and (d) obtain a probabilistic improved biopolymer sequence from the decoder. In some embodiments, the embedding is a continuously differentiable functional space representing the function and having one or more gradients. In some embodiments, calculating the change of the function with regard to the embedding comprises taking a derivative of the function with regard to the embedding. In some embodiments, the function is a composite function of two or more component functions. In some embodiments, the composite function is a weighted sum of the two or more composite functions. In some embodiments, two or more starting points in the embedding are used concurrently, e.g., at least two. In certain embodiments, 2, 3, 4, 5, 6, 7, 8, 9, 10, 20, 30, 40, 50, 100, or 200 can be used, however this is a non-limiting list. In some embodiments, correlations between residues in a probabilistic sequence comprising a probability distribution of residue identities are considered in a sampling process using conditional probabilities that account for the portion of the sequence that has already been generated. In some embodiments, the processor is further configured to select the maximum likelihood improved biopolymer sequence from a probabilistic biopolymer sequence comprising a probability distribution of residue identities. In some embodiments, the processor is further configured to sample the marginal distribution at each residue of a probabilistic biopolymer sequence comprising a probability distribution of residue identities. In some embodiments, the change of the function with regard to the embedding, is calculated by calculating the change of the function with regard to the encoder, then the change of the encoder to the change of the decoder, and the change of the decoder with regard to the embedding. In some embodiments, the processor is further configured to: provide the first updated point in the functional space or further updated point in the functional space to the decoder network to provide an intermediate probabilistic biopolymer sequence, provide the intermediate probabilistic biopolymer sequence to the supervised model network to predict the function of the intermediate probabilistic biopolymer sequence, then calculate the change in the function with regard to the embedding for the intermediate probabilistic biopolymer to provide a further updated point in the functional space.

Described herein is a non-transitory computer readable medium comprising instructions that, upon execution by a processor, cause the processor to: (a) calculate a change in the function with regard to an embedding at a starting point according to a step size, thereby providing a first updated point in the functional space, wherein the starting point is provided to a system comprising a supervised model that predicts the function of a biopolymer sequence and a decoder network, the supervised model network comprising an encoder network providing the embedding of biopolymer sequences in a functional space representing the function and the decoder network trained to provide a probabilistic biopolymer sequence, given an embedding of a biopolymer sequence in the functional space, optionally wherein the starting point is the embedding of a seed biopolymer sequence; (b) optionally calculate a change in the function with regard to the embedding at the first updated point in the functional space and optionally iterating the process of calculating a change in the function with regard to the embedding at a further updated point; (c) upon approaching a desired level of the function at the first updated point in the functional space, or optionally iterated further updated point, provide the first updated point, or optionally iterated further updated point to the decoder network; and (d) obtain a probabilistic improved biopolymer sequence from the decoder. In some embodiments, the embedding is a continuously differentiable functional space representing the function and having one or more gradients. In some embodiments, calculating the change of the function with regard to the embedding comprises taking a derivative of the function with regard to the embedding. In some embodiments, the function is a composite function of two or more component functions. In some embodiments, the composite function is a weighted sum of the two or more composite functions. In some embodiments, two or more starting points in the embedding are used concurrently, e.g., at least two. In embodiments, 2, 3, 4, 5, 6, 7, 8, 9, 10, 20, 30, 40, 50, 100, or 200 starting points can be used, although this is a non-limiting list. In some embodiments, correlations between residues in a probabilistic sequence comprising a probability distribution of residue identities are considered in a sampling process using conditional probabilities that account for the portion of the sequence that has already been generated. In some embodiments, the processor is further configured to select the maximum likelihood improved biopolymer sequence from a probabilistic biopolymer sequence comprising a probability distribution of residue identities. In some embodiments, the processor is further configured to sample the marginal distribution at each residue of a probabilistic biopolymer sequence comprising a probability distribution of residue identities. In some embodiments, the change of the function with regard to the embedding, is calculated by calculating the change of the function with regard to the encoder, then the change of the encoder to the change of the decoder, and the change of the decoder with regard to the embedding. In some embodiments, the processor is further configured to: provide the first updated point in the functional space or further updated point in the functional space to the decoder network to provide an intermediate probabilistic biopolymer sequence, provide the intermediate probabilistic biopolymer sequence to the supervised model network to predict the function of the intermediate probabilistic biopolymer sequence, then calculate the change in the function with regard to the embedding for the intermediate probabilistic biopolymer to provide a further updated point in the functional space.

Disclosed herein is a method of engineering an improved biopolymer sequence as assessed by a function, comprising: (a) predicting the function of a starting point in an embedding, the starting point provided to a system comprising a supervised model network that predicts the function of a biopolymer sequence and a decoder network, the supervised model network comprising an encoder network providing the embedding of biopolymer sequences in a functional space representing the function and the decoder network trained to provide a predicted probabilistic biopolymer sequence, optionally wherein the starting point is the embedding of a seed biopolymer sequence; (b) calculating a change in the function with regard to the embedding at the starting point according to a step size, thereby providing a first updated point in the functional space; (c) calculating, at the decoder network, a first intermediate probabilistic biopolymer sequence, based on the first updated point in the functional space; (d) predicting, at the supervised model, the function of the first intermediate probabilistic biopolymer sequence, based on the first intermediate probabilistic biopolymer sequence, (e) calculating the change in the function with regard to the embedding at the first updated point in the functional space to provide a updated point in the functional space; (f) calculating an additional intermediate probabilistic biopolymer sequence at the decoder network based on the updated point in the functional space; (g) predicting, by the supervised model, the function of the additional intermediate probabilistic biopolymer sequence based on the additional intermediate probabilistic biopolymer sequence; (h) calculating the change in the function with regard to the embedding at the further first updated point in the functional space to provide a yet further updated point in the functional space, optionally iterating steps (g)-(i), where a yet further updated point in the functional space referenced in step (h) is regarded as the further updated point in the functional space in step (f); and (i) upon approaching a desired level of the function in the functional space, providing the point in the embedding to the decoder network; and obtaining a probabilistic improved biopolymer sequence from the decoder. In some embodiments, the biopolymer is a protein. In some embodiments, the seed biopolymer sequence is an average of a plurality of sequences. In some embodiments, the seed biopolymer sequence is has no function or a level of function that is lower than the desired level of function. In some embodiments, the encoder is trained using a training data set of at least 20, 30, 40, 50, 60, 70, 80, 90, 100, 150, or 200 biopolymer sequences. In some embodiments, the encoder is a convolutional neural network (CNN) or a recurrent neural network (RNN). In some embodiments, the encoder is a transformer neural network. In some embodiments, the encoder comprises one or more convolutional layers, pooling layers, fully connected layers, normalization layers, or any combination thereof. In some embodiments, the encoder is a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the encoder comprises at least 10, 50, 100, 250, 500, 750, or 1000, or more layers. In some embodiments, the encoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the encoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the encoder is trained using a transfer learning procedure. In some embodiments, the transfer learning procedure comprises training a first model using a first biopolymer sequence training data set that is not labeled with respect to function, generating a second model comprising at least a portion of the first model, and training the second model using a second biopolymer sequence training data set that is labeled with respect to function, thereby generating the trained encoder. In some embodiments, the decoder is trained using a training data set of at least 20, 30, 40, 50, 60, 70, 80, 90, 100, 150, or 200 biopolymer sequences. In some embodiments, the decoder is a convolutional neural network (CNN) or a recurrent neural network (RNN). In some embodiments, the decoder is a transformer neural network. In some embodiments, the decoder comprises one or more convolutional layers, pooling layers, fully connected layers, normalization layers, or any combination thereof. In some embodiments, the decoder is a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the decoder comprises at least 10, 50, 100, 250, 500, 750, or 1000 layers. In some embodiments, the decoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the decoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the decoder is trained using a transfer learning procedure. In some embodiments, the transfer learning procedure comprises training a first model using first biopolymer sequence training data set that is not labeled with respect to function, generating a second model comprising at least a portion of the first model, and training the second model using a second biopolymer sequence training data set that is labeled with respect to function, thereby generating the trained decoder. In some embodiments, the one or more functions of the improved biopolymer sequence are improved compared to the one or more functions of the seed biopolymer sequence. In some embodiments, the one or more functions are selected from fluorescence, enzymatic activity, nuclease activity, and protein stability. In some embodiments, a weighted linear combination of two or more functions is used to assess the biopolymer sequence.

Described herein is a computer system comprising a processor; and a non-transitory computer readable medium encoded with software configured to cause the processor to: (a) calculate a change in the function with regard to an embedding at a starting point according to a step size, thereby providing a first updated point in the functional space, the starting point in the embedding provided to a system comprising a supervised model network that predicts the function of a biopolymer sequence and a decoder network, the supervised model network comprising an encoder network providing the embedding of biopolymer sequences in a functional space representing the function and the decoder network trained to provide a predicted probabilistic biopolymer sequence, given an embedding of the predicted biopolymer sequence in the functional space, optionally wherein the starting point is the embedding of a seed biopolymer sequence; (b) calculate a first intermediate probabilistic biopolymer sequence at the decoder network based on the first updated point in the functional space; (c) predict, at the supervised model, the function of the first intermediate probabilistic biopolymer sequence based on the first intermediate probabilistic biopolymer sequence, (d) calculate the change in the function with regard to the embedding at the first updated point in the functional space to provide a updated point in the functional space; (e) calculate, at the decoder network, an additional intermediate probabilistic biopolymer sequence based on the updated point in the functional space; (f) predict, at the supervised model, the function of the additional intermediate probabilistic biopolymer sequence based on the additional intermediate probabilistic biopolymer sequence; (g) calculate the change in the function with regard to the embedding at the further first updated point in the functional space to provide a yet further updated point in the functional space, optionally iterating steps (f)-(g), where a yet further updated point in the functional space referenced in step (g) is regarded as the further updated point in the functional space in step (e); and (i) upon approaching a desired level of the function in the functional space, provide the point in the embedding to the decoder network; and (j) obtain a probabilistic improved biopolymer sequence from the decoder. In some embodiments, the biopolymer is a protein. In some embodiments, the seed biopolymer sequence is an average of a plurality of sequences. In some embodiments, the seed biopolymer sequence is has no function or a level of function that is lower than the desired level of function. In some embodiments, the encoder is trained using a training data set of at least 20, 30, 40, 50, 60, 70, 80, 90, 100, 150, or 200 biopolymer sequences. In some embodiments, the encoder is a convolutional neural network (CNN) or a recurrent neural network (RNN). In some embodiments, the encoder is a transformer neural network. In some embodiments, the encoder comprises one or more convolutional layers, pooling layers, fully connected layers, normalization layers, or any combination thereof. In some embodiments, the encoder is a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the encoder comprises at least 10, 50, 100, 250, 500, 750, or 1000, or more layers. In some embodiments, the encoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the encoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the encoder is trained using a transfer learning procedure. In some embodiments, the transfer learning procedure comprises training a first model using a first biopolymer sequence training data set that is not labeled with respect to function, generating a second model comprising at least a portion of the first model, and training the second model using a second biopolymer sequence training data set that is labeled with respect to function, thereby generating the trained encoder. In some embodiments, the decoder is trained using a training data set of at least 20, 30, 40, 50, 60, 70, 80, 90, 100, 150, or 200 biopolymer sequences. In some embodiments, the decoder is a convolutional neural network (CNN) or a recurrent neural network (RNN). In some embodiments, the decoder is a transformer neural network. In some embodiments, the decoder comprises one or more convolutional layers, pooling layers, fully connected layers, normalization layers, or any combination thereof. In some embodiments, the decoder is a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the decoder comprises at least 10, 50, 100, 250, 500, 750, or 1000, or more layers. In some embodiments, the decoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the decoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the decoder is trained using a transfer learning procedure. In some embodiments, the transfer learning procedure comprises training a first model using first biopolymer sequence training data set that is not labeled with respect to function, generating a second model comprising at least a portion of the first model, and training the second model using a second biopolymer sequence training data set that is labeled with respect to function, thereby generating the trained decoder. In some embodiments, the one or more functions of the improved biopolymer sequence are improved compared to the one or more functions of the seed biopolymer sequence. In some embodiments, the one or more functions are selected from fluorescence, enzymatic activity, nuclease activity, and protein stability. In some embodiments, a weighted linear combination of two or more functions is used to assess the biopolymer sequence.

Described herein is a non-transitory computer readable medium comprising instructions that, upon execution by a processor, cause the processor to: (a) predict the function of a starting point in an embedding, wherein the starting point is the embedding of a seed biopolymer sequence, the starting point provided to a system comprising a supervised model network that predicts the function of a biopolymer sequence and a decoder network, the supervised model network comprising an encoder network providing the embedding of biopolymer sequences in a functional space representing the function and the decoder network trained to provide a predicted probabilistic biopolymer sequence, given an embedding of the predicted biopolymer sequence in the functional space; (b) calculate a change in the function with regard to the embedding at the starting point according to a step size, thereby providing a first updated point in the functional space; (c) provide the first updated point in the functional space to the decoder network to provide a first intermediate probabilistic biopolymer sequence; (d) predict the function of the first intermediate probabilistic biopolymer sequence, by the supervised model, based on the first intermediate probabilistic biopolymer sequence, (e) calculate the change in the function with regard to the embedding at the first updated point in the functional space to provide a updated point in the functional space; (f) provide an additional intermediate probabilistic biopolymer sequence by the decoder network based on updated point in the functional space; (g) predict the function of the additional intermediate probabilistic biopolymer sequence provide the additional intermediate probabilistic biopolymer sequence to the supervised model to; (h) calculate the change in the function with regard to the embedding at the further first updated point in the functional space to provide a yet further updated point in the functional space, optionally iterating steps (f)-(h), where a yet further updated point in the functional space referenced in step (h) is regarded as the further updated point in the functional space in step (f); and (i) upon approaching a desired level of the function in the functional space, provide the point in the embedding to the decoder network; and obtain a probabilistic improved biopolymer sequence from the decoder. In some embodiments, the biopolymer is a protein. In some embodiments, the seed biopolymer sequence is an average of a plurality of sequences. In some embodiments, the seed biopolymer sequence is has no function or a level of function that is lower than the desired level of function. In some embodiments, the encoder is trained using a training data set of at least 20, 30, 40, 50, 60, 70, 80, 90, 100, 150, or 200 biopolymer sequences. In some embodiments, the encoder is a convolutional neural network (CNN) or a recurrent neural network (RNN). In some embodiments, the encoder is a transformer neural network. In some embodiments, the encoder comprises one or more convolutional layers, pooling layers, fully connected layers, normalization layers, or any combination thereof. In some embodiments, the encoder is a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the encoder comprises at least 10, 50, 100, 250, 500, 750, or 1000, or more layers. In some embodiments, the encoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the encoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the encoder is trained using a transfer learning procedure. In some embodiments, the transfer learning procedure comprises training a first model using a first biopolymer sequence training data set that is not labeled with respect to function, generating a second model comprising at least a portion of the first model, and training the second model using a second biopolymer sequence training data set that is labeled with respect to function, thereby generating the trained encoder. In some embodiments, the decoder is trained using a training data set of at least 20, 30, 40, 50, 60, 70, 80, 90, 100, 150, or 200 biopolymer sequences. In some embodiments, the decoder is a convolutional neural network (CNN) or a recurrent neural network (RNN). In some embodiments, the decoder is a transformer neural network. In some embodiments, the decoder comprises one or more convolutional layers, pooling layers, fully connected layers, normalization layers, or any combination thereof. In some embodiments, the decoder is a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the decoder comprises at least 10, 50, 100, 250, 500, 750, or 1000, or more layers. In some embodiments, the decoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the decoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the decoder is trained using a transfer learning procedure. In some embodiments, the transfer learning procedure comprises training a first model using first biopolymer sequence training data set that is not labeled with respect to function, generating a second model comprising at least a portion of the first model, and training the second model using a second biopolymer sequence training data set that is labeled with respect to function, thereby generating the trained decoder. In some embodiments, the one or more functions of the improved biopolymer sequence are improved compared to the one or more functions of the seed biopolymer sequence. In some embodiments, the one or more functions are selected from fluorescence, enzymatic activity, nuclease activity, and protein stability. In some embodiments, a weighted linear combination of two or more functions is used to assess the biopolymer sequence.

Disclosed herein is a computer implemented method for engineering a biopolymer sequence having a specified protein function, comprising: (a) generating, with an encoder method, an embedding of an initial biopolymer sequence; (b) iteratively changing, with an optimization method, the embedding to correspond to the specified protein function by adjusting one or more embedding parameters, thereby generating an updated embedding; (c) processing, by a decoder method, the updated embedding to generate a final biopolymer sequence. In some embodiments, the biopolymer sequence comprises a primary protein amino acid sequence. In some embodiments, the amino acid sequence causes a protein configuration that results in the protein function. In some embodiments, the protein function comprises fluorescence. In some embodiments, the protein function comprises an enzymatic activity. In some embodiments, the protein function comprises nuclease activity. In some embodiments, the protein function comprises a degree of protein stability. In some embodiments, the encoder method is configured to receive the initial biopolymer sequence and generate the embedding. In some embodiments, the encoder method comprises a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the encoder comprises at least 10, 50, 100, 250, 500, 750, or 1000, or more layers. In some embodiments, the encoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the encoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the decoder method comprises a deep convolutional neural network. In some embodiments, a weighted linear combination of two or more functions is used to assess the biopolymer sequence. In some embodiments, the optimization method generates the updated embedding using gradient-based descent within the continuous and differentiable embedding space. In some embodiments, the optimization method utilizes an optimization scheme selected from Adam, RMS Prop, Ada delta, AdamMAX, or SGD with momentum. In some embodiments, the final biopolymer sequence is further optimized for at least one additional protein function. In some embodiments, the optimization method generates the updated embedding according to a composite function integrating both the protein function and the at least one additional protein function. In some embodiments, the composite function is a weighted linear combination of two or more functions corresponding to the protein function and the at least one additional protein function.

Disclosed herein is a computer implemented method for engineering a biopolymer sequence having a specified protein function, comprising: (a) generating, with an encoder method, an embedding of an initial biopolymer sequence; (b) adjusting, with an optimization method, the embedding by modifying one or more embedding parameters to achieve the specified protein function, thereby generating an updated embedding; (c) processing, by a decoder method, the updated embedding to generate a final biopolymer sequence.

Described herein is a computer system comprising a processor; and a non-transitory computer readable medium encoded with software configured to cause the processor to: (a) generate, with an encoder method, an embedding of an initial biopolymer sequence; (b) iteratively change, with an optimization method, the embedding to correspond to the specified protein function by adjusting one or more embedding parameters, thereby generating an updated embedding; (c) process, by a decoder method, the updated embedding to generate a final biopolymer sequence. In some embodiments, the biopolymer sequence comprises a primary protein amino acid sequence. In some embodiments, the amino acid sequence causes a protein configuration that results in the protein function. In some embodiments, the protein function comprises fluorescence. In some embodiments, the protein function comprises an enzymatic activity. In some embodiments, the protein function comprises nuclease activity. In some embodiments, the protein function comprises a degree of protein stability. In some embodiments, the encoder method is configured to receive the initial biopolymer sequence and generate the embedding. In some embodiments, the encoder method comprises a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the encoder comprises at least 10, 50, 100, 250, 500, 750, or 1000, or more layers. In some embodiments, the encoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the encoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the decoder method comprises a deep convolutional neural network. In some embodiments, a weighted linear combination of two or more functions is used to assess the biopolymer sequence. In some embodiments, the optimization method generates the updated embedding using gradient-based descent within the continuous and differentiable embedding space. In some embodiments, the optimization method utilizes an optimization scheme selected from Adam, RMS Prop, Ada delta, AdamMAX, or SGD with momentum. In some embodiments, the final biopolymer sequence is further optimized for at least one additional protein function. In some embodiments, the optimization method generates the updated embedding according to a composite function integrating both the protein function and the at least one additional protein function. In some embodiments, the composite function is a weighted linear combination of two or more functions corresponding to the protein function and the at least one additional protein function.

Described herein is a non-transitory computer readable medium comprising instructions that, upon execution by a processor, cause the processor to: (a) generate, with an encoder method, an embedding of an initial biopolymer sequence; (b) iteratively change, with an optimization method, the embedding to correspond to the specified protein function by adjusting one or more embedding parameters, thereby generating an updated embedding; (c) process, by a decoder method, the updated embedding to generate a final biopolymer sequence. In some embodiments, the biopolymer sequence comprises a primary protein amino acid sequence. In some embodiments, the amino acid sequence causes a protein configuration that results in the protein function. In some embodiments, the protein function comprises fluorescence. In some embodiments, the protein function comprises an enzymatic activity. In some embodiments, the protein function comprises nuclease activity. In some embodiments, the protein function comprises a degree of protein stability. In some embodiments, the encoder method is configured to receive the initial biopolymer sequence and generate the embedding. In some embodiments, the encoder method comprises a deep convolutional neural network. In some embodiments, the convolutional neural network is a one-dimensional convolutional network. In some embodiments, the convolutional neural network is a two-dimensional, or higher, convolutional neural network. In some embodiments, the convolutional neural network has a convolutional architecture selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the encoder comprises at least 10, 50, 100, 250, 500, 750, or 1000, or more layers. In some embodiments, the encoder employs a regularization method comprising L1-L2 regularization on one or more layers, skip connections on one or more layers, drop outs at one or more layers, or a combination thereof. In some embodiments, the regularization is performed using batch normalization. In some embodiments, the regularization is performed using group normalization. In some embodiments, the encoder is optimized by a procedure selected from Adam, RMS prop, stochastic gradient descent (SGD) with momentum, SGD with momentum and Nestrop, SGD without momentum, Adagrad, Adadelta, or NAdam. In some embodiments, the decoder method comprises a deep convolutional neural network. In some embodiments, a weighted linear combination of two or more functions is used to assess the biopolymer sequence. In some embodiments, the optimization method generates the updated embedding using gradient-based descent within the continuous and differentiable embedding space. In some embodiments, the optimization method utilizes an optimization scheme selected from Adam, RMS Prop, Ada delta, AdamMAX, or SGD with momentum. In some embodiments, the final biopolymer sequence is further optimized for at least one additional protein function. In some embodiments, the optimization method generates the updated embedding according to a composite function integrating both the protein function and the at least one additional protein function. In some embodiments, the composite function is a weighted linear combination of two or more functions corresponding to the protein function and the at least one additional protein function.

Disclosed herein is a method of making a biopolymer comprising synthesizing an improved biopolymer sequence obtainable by a method of any one of the preceding embodiments or using a system of any one of the preceding embodiments.

Disclosed herein is a fluorescent protein comprising an amino acid sequence, relative to SEQ ID NO:1, that includes a substitution at a site selected from Y39, F64, V68, D129, V163, K166, G191, or a combination thereof, and having increased fluorescence, relative to SEQ ID NO:1. In some embodiments, the fluorescent protein comprises substitutions at 2, 3, 4, 5, 6, or all 7 of Y39, F64, V68, D129, V163, K166, and G191. In some embodiments, the fluorescent protein comprises, relative to SEQ ID NO:1, S65. In some embodiments, the amino acid sequence comprises, relative to SEQ ID NO:1, S65. In some embodiments, the amino acid sequence comprises substitutions at F64 and V68. In some embodiments, the amino acid sequence comprises 1, 2, 3, 4, or all 5 of Y39, D129, V163, K166, and G191. In some embodiments, the substitutions at Y39, F64, V68, D129, V163, K166, or G191 are Y39C, F64L, V68M, D129G, V163A, K166R, or G191V, respectively. In some embodiments, the fluorescent protein comprises an amino acid sequence at least 80, 85, 90, 92, 92, 93, 94, 95, 96, 97, 98, 99%, or more, identical to SEQ ID NO:1. In some embodiments, the fluorescent protein comprises, relative to SEQ ID NO:1, at least 1, 2, 3, 4, 5, 6, 7, 8, 9, 10, 11, 12, 13, 14, or 15 mutations. In some embodiments, the fluorescent protein comprises, relative to SEQ ID NO:1, no more than 1, 2, 3, 4, 5, 6, 7, 8, 9, 10, 11, 12, 13, 14, or 15 mutations. In some embodiments, the fluorescent protein has at least about: 2, 3, 4, 5, 6, 7, 8, 9, 10, 15, 20, 25, 30, 35, 40, 45, or 50-fold greater fluorescence intensity than SEQ ID NO:1. In some embodiments, the fluorescent protein has at least about: 2, 3, 4, or 5-fold greater fluorescence than super-folder GFP (AIC82357). In some embodiments, disclosed herein is a fusion protein comprising the fluorescent protein. In some embodiments, disclosed herein is a nucleic acid comprising a sequence encoding the fluorescent protein or fusion protein. In some embodiments, disclosed herein is a vector comprising the nucleic acid. In some embodiments, disclosed herein is a host cell comprising the protein, the nucleic acid, or the vector. In some embodiments, disclosed herein is a method of visualization, comprising detecting the fluorescent protein. In some embodiments, the detection is by detecting a wavelength of the emission spectrum of the fluorescent protein. In some embodiments, the visualization is in a cell. In some embodiments, the cell is in an isolated biological tissue, in vitro, or in vivo. In some embodiments, disclosed herein is a method of expressing the fluorescent protein or fusion protein, comprising introducing an expression vector comprising a nucleic acid encoding the polypeptide into a cell. In some embodiments, the method further comprises culturing the cell to grow a batch of cultured cells and purifying the polypeptide from the batch of cultured cells. In some embodiments, disclosed herein is a method of detecting a fluorescent signal of a polypeptide inside a biological cell or tissue, tissue, comprising: (a) introducing the fluorescent protein or an expression vector comprising a nucleic acid encoding said fluorescent protein into the biological cell or tissue; (b) directing a first wavelength of light suitable for exciting the fluorescent protein at the biological cell or tissue; and (c) detecting a second wavelength of light emitted by the fluorescent protein in response to absorption of the first wavelength of light. In some embodiments, the second wavelength of light is detected using a fluorescence microscope or fluorescence activated cell sorting (FACS). In some embodiments, the biological cell or tissue is a prokaryotic or eukaryotic cell. In some embodiments, the expression vector comprises a fusion gene comprising the nucleic acid encoding the polypeptide fused to another gene on the N- or C-terminus. In some embodiments, the expression vector comprises a promoter controlling expression of the polypeptide that is a constitutively active promoter or an inducible expression promoter.

Disclosed is a method for training a supervised model for use in a method or system as described before. This supervised model comprises an encoder network that is configured to map biopolymer sequences to representations in an embedding functional space. The supervised model is configured to predict a function of the biopolymer sequence based on the representations. The method comprises the steps of: (a) providing a plurality of training biopolymer sequences, wherein each training biopolymer sequence is labelled with a function; (b) mapping, using the encoder, each training biopolymer sequence to a representation in the embedding functional space; (c) predicting, using the supervised model, based on these representations, the function of each training biopolymer sequence; (d) determining, using a predetermined prediction loss function, for each training biopolymer sequence, how well the predicted function is in agreement with the function as per the label of the respective training biopolymer sequence; and (e) optimizing parameters that characterize the behavior of the supervised model with the goal of improving the rating by said prediction loss function that results when further training biopolymer sequences are processed by the supervised model.

Disclosed is a method for training a decoder for use in a method or system as described before. The decoder is configured to map a representation of a biopolymer sequence from an embedding functional space to a probabilistic biopolymer sequence. The method comprises the steps of: (a) providing a plurality of representations of biopolymer sequences in the embedding functional space; (b) mapping, using the decoder, each representation to a probabilistic biopolymer sequence; (c) drawing a sample biopolymer sequence from each probabilistic biopolymer sequence; (d) mapping, using a trained encoder, this sample biopolymer sequence to a representation in said embedding functional space; (e) determining, using a predetermined reconstruction loss function, how well each so-determined representation is in agreement with the corresponding original representation; and (f) optimizing parameters that characterize the behavior of the decoder with the goal of improving the rating by said reconstruction loss function that results when further representations of biopolymer sequences from said embedding functional space are processed by the decoder.

Optionally, the encoder is part of a supervised model that is configured to predict a function of the biopolymer sequence based on the representations generated by the decoder, and the method further comprises: (a) providing at least part of the plurality of representations of biopolymer sequences to the decoder by mapping training biopolymer sequences to representations in the embedding functional space using the trained encoder; (b) predicting, for the sample biopolymer sequence drawn from the probabilistic biopolymer sequence, using the supervised model, a function of this sample biopolymer sequence; (c) comparing said function to a function predicted by the same supervised model for the corresponding original training biopolymer sequence; (d) determining, using a predetermined consistency loss function, how well the function predicted for the sample biopolymer sequence is in agreement with the function predicted for the original training biopolymer sequence; and (e) optimizing parameters that characterize the behavior of the decoder with the goal of improving the rating by said consistency loss function, and/or by a predetermined combination of said consistency loss function with said reconstruction loss function, that results when further representations of biopolymer sequences generated by the encoder from training biopolymer sequences are processed by the decoder.

Disclosed is a method for training an ensemble of a supervised model and a decoder. The supervised model comprises an encoder network that is configured to map biopolymer sequences to representations in an embedding functional space. The supervised model is configured to predict a function of the biopolymer sequence based on the representations. The decoder is configured to map a representation of a biopolymer sequence from an embedding functional space to a probabilistic biopolymer sequence. The method comprises the steps of: (a) providing a plurality of training biopolymer sequences, wherein each training biopolymer sequence is labelled with a function; (b) mapping, using the encoder, each training biopolymer sequence to a representation in the embedding functional space; (c) predicting, using the supervised model, based on these representations, the function of each training biopolymer sequence; (d) mapping, using the decoder, each representation in the embedding functional space to a probabilistic biopolymer sequence; (e) drawing a sample biopolymer sequence from the probabilistic biopolymer sequence; (f) determining, using a predetermined prediction loss function, for each training biopolymer sequence, how well the predicted function is in agreement with the function as per the label of the respective training biopolymer sequence; (g) determining, using a predetermined reconstruction loss function, for each sample biopolymer sequence, how well it is in agreement with the original training biopolymer sequence from which it was produced; and (h) optimizing parameters that characterize the behavior of the supervised model and parameters that characterize the behavior of the decoder with the goal of improving the rating by a predetermined combination of the prediction loss function and the reconstruction loss function.

Furthermore, a set of parameters that characterize the behavior of a supervised model, an encoder or a decoder obtained according to one of these training methods is another product within the scope of the present invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Specifically, U.S. Application No. 62/804,036 is herein incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings of which:

FIG. 1 shows a diagram illustrating a non-limiting embodiment of the encoder as a neural network.

FIG. 2 shows a diagram illustrating a non-limiting embodiment of the decoder as a neural network.

FIG. 3A shows a non-limiting overview of a gradient-based design procedure.

FIG. 3B shows a non-limiting example of one iteration of a gradient-based design procedure.

FIG. 3C shows a non-limiting example of a matrix encoding a probabilistic sequence generated by a decoder.

FIG. 4 shows a diagram illustrating a non-limiting embodiment of a decoder validation procedure.

FIG. 5A shows a graph of the predicted vs. true fluorescence values from a GFP encoder model for a training data set.

FIG. 5B shows a graph of the predicted vs. true fluorescence values from the GFP encoder model for a validation data set.

FIG. 6A-B shows an exemplary embodiment of a computing system as described herein.

FIG. 7 shows a diagram illustrating a non-limiting example of gradient-based design (GBD) for engineering a GFP sequence.

FIG. 8 shows experimental validation results with relative fluorescence values for GFP sequences created using GBD.

FIG. 9 shows a pairwise amino acid sequence alignment of avGFP against the GBD-engineered GFP sequence with the highest experimentally validated fluorescence.

FIG. 10 shows a chart illustrating the evolution of the predicted resistance through rounds or iterations of gradient-based design.

FIG. 11 shows the results of a validation experiment performed to assess the actual antibiotic resistance conferred by seven novel beta-lactamases designed using gradient-based design.

FIG. 12A-F are graphs illustrating discrete optimization results on RNA optimization (12A-C) and lattice-protein optimization (12D-F).

FIGS. 13A-H is a diagram illustrating results for gradient-based optimization.

FIGS. 14A-B is a diagram illustrating the effect of up-weighting the regularization term λ: larger λ results in decreased model error but a corresponding decrease in sequence diversity over the course of optimization as the model is restricted to sequences that are assigned high probability by p_{θ}.

FIGS. 15A-B illustrates the heuristic motivating GBD: it drives the cohort to areas of Z where d*_{φ} can decode reliably.

FIG. 16 illustrates that GBD is able to find optima further away from initial seed sequences than discrete methods while maintaining a comparably low error.

FIG. 17 is a graph illustrating wet lab data testing the generated variance of the listed proteins, validating the affinity of the generated proteins.

### DETAILED DESCRIPTION

Described herein are systems, apparatuses, software, and methods for generating predictions of amino acid sequences corresponding to properties or functions. Machine learning methods allow for the generation of models that receive input data such as a primary amino acid sequence and generating a modified amino acid sequence corresponding to one or more functions or features of the resulting polypeptide or protein defined at least in part by the amino acid sequence. The input data can include additional information such as contact maps of amino acid interactions, tertiary protein structure, or other relevant information relating to the structure of the polypeptide. Transfer learning is used in some instances to improve the predictive ability of the model when there is insufficient labeled training data. The input amino acid sequence can be mapped into an embedding space, optimized within the embedding space with respect to a desired function or property (e.g., increasing reaction rate of an enzyme), and then decoded into a modified amino acid sequence that maps to the desired function or property.

The present disclosure incorporates the novel discovery that proteins are amenable to machine learning-based rational sequence design, such as gradient-based design using deep neural networks, which allows standard optimization techniques to be used (e.g., gradient ascent) to create sequences of amino acids that perform the desired function. In the illustrative example of gradient-based design, an initial sequence of amino acids is projected into a new embedding space which is representative of the protein's function. An embedding of the protein sequence is a representation of a protein as a point in D-dimensional space. In this new space, a protein can be encoded as a vector of two numbers (e.g., in the case of a 2-dimensional space), which provide the coordinates for that protein in the embedding space. A property of the embedding space is that proteins which are nearby in this space are functionally similar and related. Accordingly, when a collection of proteins have been embedded into this space, the similarity of function of any two proteins can be determined by computing the distance between them using a Euclidean metric.

### In Silico Protein Design

In some embodiments, the devices, software, systems, and methods disclosed herein utilize machine learning method(s) as a tool for protein design. In some embodiments, a continuous and differentiable embedding space is used to generate a novel protein or polypeptide sequence mapped to a desired function or property. In some cases, the process comprises providing a seed sequence (e.g., a sequence that does not perform the desired function(s) or does not perform the desired function at the desired level), projecting the seed sequence into the embedding space, iteratively optimizing the sequence by making small changes in embedding space, and then mapping these changes back into sequence space. In some instances, the seed sequence lacks the desired function or property (e.g., beta-lactamase having no antibiotic resistance). In some cases, the seed sequence has some function or property (e.g., a baseline GFP sequence having some fluorescence). The seed sequence can have the highest or “best” available function or property (e.g., the GFP having the highest fluorescence intensity from the literature). The seed sequence may have the closest function or property to a desired function or property. For example, a seed GFP sequence can be selected that is has the fluorescence intensity value that is closest to a final desired fluorescence intensity value. The seed sequence can be based on a single sequence or an average or consensus sequence of a plurality of sequences. For example, multiple GFP sequences can be averaged to produce a consensus sequence. The sequences that are averaged may represent a starting point of the “best” sequences, (e.g., those having the highest or closest level of the desired function or property that is to be optimized). The approach disclosed herein can utilize more than one method or trained model. In some embodiments, two neural networks are provided that work in tandem: an encoder network and a decoder network. The encoder network can receive a sequence of amino acids, which may be represented as a sequence of one-hot vectors, and generate the embedding for that protein. Likewise, the decoder can obtain the embedding and return the sequence of amino acids that maps to a particular point in the embedding space.

To change a given protein's function, the initial sequence can be first projected into the embedding space using the encoder network. Next, the protein function can be changed by “moving” the initial sequence's position within the embedding space towards the region of space occupied by proteins that have the desired function (or level of function, e.g., enhanced function). Once the embedded sequence has moved to the desired region of embedding space (and thus achieved the desired level of function), the decoder network can be used to receive the new coordinates in embedding space and produce the actual sequence of amino acids that would encode a real protein having the desired function or level of function. In some embodiments, in which the encoder and decoder networks are deep neural networks, partial derivatives can be computed for points within the embedding space, thus allowing optimization methods such as, for example, gradient based optimization procedures to compute directions of steepest improvement in this space.

A simplified, step-by-step overview of one embodiment of the in silico protein design described herein includes the following steps:

(1) Select a protein to serve as a “seed” protein. This protein serves as the base sequence to be modified.

(2) Project this protein into embedding space using the encoder network.

(3) Perform iterative improvements on the seed protein within the embedding space using a gradient ascent procedure, which is based on the derivative of the function with respect to the embedding provided by the encoder network.

(4) Once the desired level of function is obtained, map the final embedding back into sequence space using the decoder network. This produces the sequence of amino acids with the desired level of functionality.

Construction of the Embedding Space

In some embodiments, the devices, software, systems, and methods disclosed herein utilize an encoder to generate an embedding space when given an input such as a primary amino acid sequence. In some embodiments, the encoder is constructed by training a neural network (e.g., a deep neural network) to predict the desired function based on a set of labeled training data. The encoder model can be a supervised model using a convolutional neural network (CNN) in the form of a 1D convolution (e.g., primary amino acid sequence), a 2D convolution (e.g., contact maps of amino acid interactions), or a 3D convolution (e.g., tertiary protein structures). The convolutional architecture can be any of the following described architectures: VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet.

In some embodiments, the encoder utilizes any number of alternative regularization methods to prevent overfitting. Illustrative and non-limiting examples of regularization methods includes early stopping, including drop outs at least at 1, 2, 3, 4, up to all layers, including L1-L2 regularization on at least 1, 2, 3, 4, up to all layers, including skip connections at least at 1, 2, 3, 4, up to all layers. Herein, the term “drop out” may in particular comprise randomly deactivating some of the neurons or other processing units of the layer during training, so that the training is in fact performed on a large number of slightly different network architectures. This reduces “overfitting”, i.e., over-adapting the network to the concrete training data at hand, rather than learning generalized knowledge from this training data. Alternatively or in combination, regularization can be performed using batch normalization or group normalization.

In some embodiments, the encoder is optimized using any of the following non-limiting optimization procedures: Adam, RMS prop, stochastic gradient descent (SGD) with momentum, SGD with momentum and Nestrop, SGD without momentum, Adagrad, Adadelta, or NAdam. A model can be optimized using any of the follow activation functions: softmax, elu, SeLU, softplus, softsign, ReLU, tan h, sigmoid, hard_sigmoid, exponential, PReLU, and LeaskyReLU, or linear.

In some embodiments, the encoder comprises 3 layers to 100,000 layers. In some embodiments, the encoder comprises 3 layers to 5 layers, 3 layers to 10 layers, 3 layers to 50 layers, 3 layers to 100 layers, 3 layers to 500 layers, 3 layers to 1,000 layers, 3 layers to 5,000 layers, 3 layers to 10,000 layers, 3 layers to 50,000 layers, 3 layers to 100,000 layers, 3 layers to 100,000 layers, 5 layers to 10 layers, 5 layers to 50 layers, 5 layers to 100 layers, 5 layers to 500 layers, 5 layers to 1,000 layers, 5 layers to 5,000 layers, 5 layers to 10,000 layers, 5 layers to 50,000 layers, 5 layers to 100,000 layers, 5 layers to 100,000 layers, 10 layers to 50 layers, 10 layers to 100 layers, 10 layers to 500 layers, 10 layers to 1,000 layers, 10 layers to 5,000 layers, 10 layers to 10,000 layers, 10 layers to 50,000 layers, 10 layers to 100,000 layers, 10 layers to 100,000 layers, 50 layers to 100 layers, 50 layers to 500 layers, 50 layers to 1,000 layers, 50 layers to 5,000 layers, 50 layers to 10,000 layers, 50 layers to 50,000 layers, 50 layers to 100,000 layers, 50 layers to 100,000 layers, 100 layers to 500 layers, 100 layers to 1,000 layers, 100 layers to 5,000 layers, 100 layers to 10,000 layers, 100 layers to 50,000 layers, 100 layers to 100,000 layers, 100 layers to 100,000 layers, 500 layers to 1,000 layers, 500 layers to 5,000 layers, 500 layers to 10,000 layers, 500 layers to 50,000 layers, 500 layers to 100,000 layers, 500 layers to 100,000 layers, 1,000 layers to 5,000 layers, 1,000 layers to 10,000 layers, 1,000 layers to 50,000 layers, 1,000 layers to 100,000 layers, 1,000 layers to 100,000 layers, 5,000 layers to 10,000 layers, 5,000 layers to 50,000 layers, 5,000 layers to 100,000 layers, 5,000 layers to 100,000 layers, 10,000 layers to 50,000 layers, 10,000 layers to 100,000 layers, 10,000 layers to 100,000 layers, 50,000 layers to 100,000 layers, 50,000 layers to 100,000 layers, or 100,000 layers to 100,000 layers. In some embodiments, the encoder comprises 3 layers, 5 layers, 10 layers, 50 layers, 100 layers, 500 layers, 1,000 layers, 5,000 layers, 10,000 layers, 50,000 layers, 100,000 layers, or 100,000 layers. In some embodiments, the encoder comprises at least 3 layers, 5 layers, 10 layers, 50 layers, 100 layers, 500 layers, 1,000 layers, 5,000 layers, 10,000 layers, 50,000 layers, or 100,000 layers. In some embodiments, the encoder comprises at most 5 layers, 10 layers, 50 layers, 100 layers, 500 layers, 1,000 layers, 5,000 layers, 10,000 layers, 50,000 layers, 100,000 layers, or 100,000 layers.

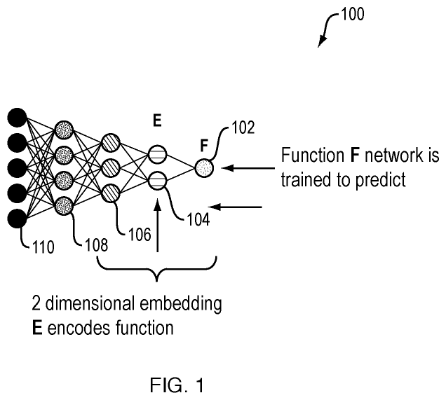

In some embodiments, the encoder is trained to predict the function or property of a protein or polypeptide given its raw sequence of amino acids. As a by-product of learning to predict, the penultimate layer of the encoder encodes the original sequence in the embedding space. Thus, to embed a given sequence, the given sequence is passed through all layers of the network up to the penultimate layer and the pattern of activations at this layer is taken as the embedding. FIG. 1 is a diagram illustrating a non-limiting embodiment of the encoder 100 as a neural network. The encoder neural network is trained to predict a specific function 102 given an input sequence 110. The penultimate layer is a two-dimensional embedding 104 that encodes all of the information about the function of a given sequence. Accordingly, an encoder can obtain an input sequence, such as a sequence of amino acids or a nucleic acid sequence corresponding to the amino acid sequence, and process the sequence to create an embedding or vectorized representation of the source sequence that captures the function of the amino acid sequence within the embedding space. The selection of initial source sequences can be based on rational means (e.g., the protein(s) with the highest level of function) or by some other means, (e.g., random selection).

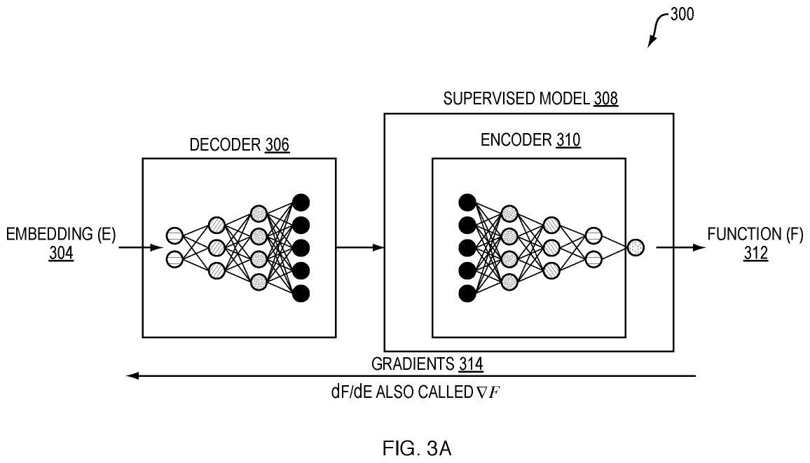

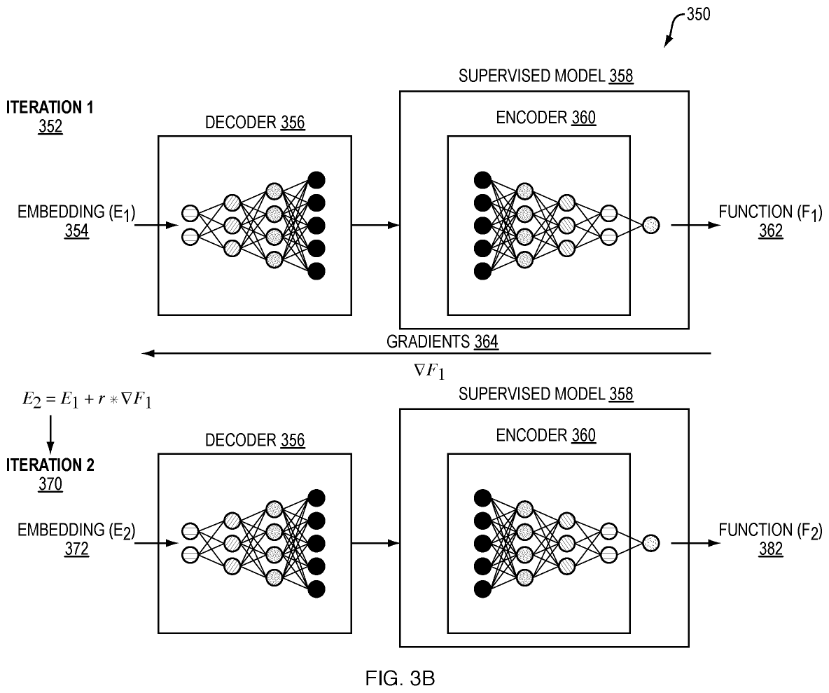

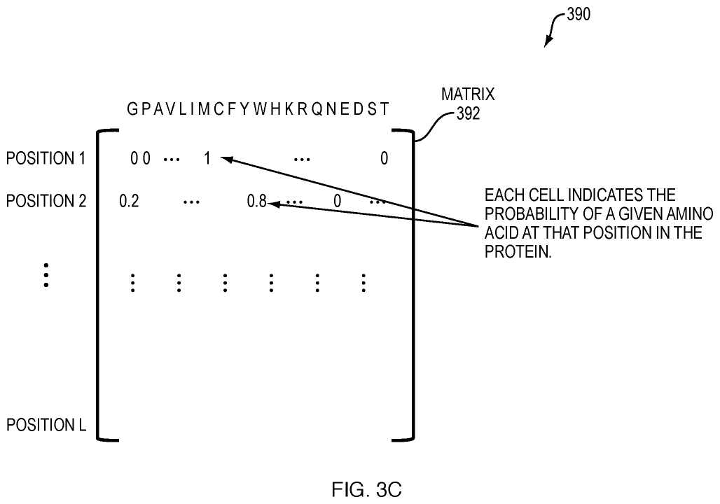

However, it is not strictly required that the encoder goes all the way from the input sequence to the concrete quantitative value of the function. Rather, a layer or other processing unit that is distinct from the encoder may take in the embedding delivered by the encoder and map this to the sought quantitative value of the function. One such embodiment is illustrated in FIG. 3A.

The encoder and the decoder may be trained at least partially in tandem in an encoder-decoder arrangement. Irrespective of whether the quantitative value of the function is evaluated within the encoder or outside the encoder, starting from an input biopolymer sequence, the compressed representation in the embedding space produced by the encoder may be fed into the decoder, and it may then be determined how well the probabilistic biopolymer sequence delivered by the decoder is in agreement with the original input biopolymer sequence. For example, one or more samples may be drawn from the probabilistic biopolymer sequence, and the one or more drawn samples may be compared to the original input biopolymer sequence. Parameters that characterize the behavior of the encoder and/or the decoder may then be optimized such that agreement between the probabilistic biopolymer sequence and the original input biopolymer sequence is maximized.

As will be discussed later, such agreement may be measured by a predetermined loss function (“reconstruction loss”). On top of that, the prediction of the function may be trained on input biopolymer sequences that are labeled with a known value of the function that should be reproduced by the prediction. The agreement of the prediction with the actual known value of the function may be measured by another loss that may be combined with said reconstruction loss in any suitable manner.

In some embodiments, the encoder is generated at least in part using transfer learning to improve performance. The starting point can be the full first model frozen except the output layer (or one or more additional layers), which is trained on the target protein function or protein feature. The starting point can be the pretrained model, in which the embedding layer, last 2 layers, last 3 layers, or all layers are unfrozen and the rest of the model is frozen during training on the target protein function or protein feature.

Gradient-Based Protein Design in Embedding Space

In some embodiments, the devices, software, systems, and methods disclosed herein obtain an initial embedding of input data such as a primary amino acid sequence and optimize the embedding towards a particular function or property. In some embodiments, once an embedding has been created, the embedding is optimized towards a given function using a mathematical method such as the ‘back-propagation’ method to compute the derivatives of the embedding with respect to the function to be optimized. Given an initial embedding E₁, a learning rate r, the gradient ∇F of the function F, the following update can be performed to create a new embedding, E₂:

$${E}_{2} = {E}_{1} + r * ∇ F$$

The gradient of F (∇F) is implicitly defined by the encoder network and due to the fact that the encoder is differentiable almost everywhere, the derivate of the embedding with respect to the function can be computed. The above update procedure can be repeated until the desired level of the function has been achieved.

FIG. 3B is a diagram illustrating iterations of gradient-based design (GBD). First, a source embedding 354 is fed into the GBD network 350 comprised of a decoder 356 and supervised model 358. The gradients 364 are computed and used to produce a new embedding which is then fed back into the GBD network 350 via decoder 356 to eventually generate function F₂ 382. This process can be repeated until a desired level of the function has been obtained or until the predicted function has saturated.

There are many possible variations for this update rule, which include different step sizes for r, and different optimization schemes, such as Adam, RMS Prop, Ada delta, AdamMAX, and SGD with momentum. Additionally, the above update is an example of a ‘first-order’ method that only uses information about the first derivative, but, in some embodiments, higher order methods such as, for example, 2^{nd}-order methods, can be utilized which leverage information contained in the Hessian.

Using the embedding optimization approaches described herein, constraints and other desired data can be incorporated as long as they can be incorporated into the update equation. In some embodiments, the embedding is optimized for at least two, at least three, at least four, at least five, at least six, at least seven, at least eight, at least nine, or at least ten parameters (e.g., desired functions and/or properties). As a non-limiting and illustrative example, a sequence is being optimized for both function F₁ (e.g., fluorescence) and function F₂ (e.g., thermostability). In this scenario, the encoder has been trained to predict both of these functions, thus allowing a composite function F=c₁F₁+c₂F₂ to be used that incorporates both functions into the optimization process, weighting the functions as desired. Accordingly, this composite function can be optimized such as using the gradient-based update procedure described herein. In some embodiments, the devices, software, systems, and methods described herein utilize a composite function that incorporates weights that express the relative preferences for F₁ and F₂ under this framework (e.g., mostly maximize fluorescence but also incorporate some thermostability).

Mapping Back to Protein Space: The Decoder Network

In some embodiments, the devices, software, systems, and methods disclosed herein obtain the seed embedding that has been optimized to achieve some desired level of function and utilize a decoder to map the optimized coordinates in the embedding space back into protein space. In some embodiments, a decoder, such as a neural network, is trained to produce the amino acid sequence based on an input comprising an embedding. This network essentially provides the “inverse” of the encoder and can be implemented using a deep convolutional neural network. In other words, an encoder receives an input amino acid sequence and generates an embedding of the sequence mapped into the embedding space, and the decoder receives input (optimized) embedding coordinates and generates a resulting amino acid sequence. The decoder can be trained using labeled data (e.g., beta-lactamases labeled with antibiotic resistance information) or unlabeled data (e.g., beta-lactamases lacking antibiotic resistance information). In some embodiments, the overall structure of the decoder and encoder are the same. For example, the number of variations (architecture, number of layers, optimizers, etc) can be the same for the decoder as it is for the encoder.

In some embodiments, the devices, software, systems, and methods disclosed herein utilize a decoder to process an input such as a primary amino acid sequence or other biopolymer sequence and generate a predicted sequence (e.g., a probabilistic sequence having a distribution of amino acids at each position). In some embodiments, the decoder is constructed by training a neural network (e.g., a deep neural network) to generate the predicted sequence based on a set of labeled training data. For example, embeddings can be generated from the labeled training data, and then used to train the decoder. The decoder model can be a supervised model using a convolutional neural network (CNN) in the form of a 1D convolution (e.g., primary amino acid sequence), a 2D convolution (e.g., contact maps of amino acid interactions), or a 3D convolution (e.g., tertiary protein structures). The convolutional architecture can be any of the following described architectures: VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet.

In some embodiments, the decoder utilizes any number of alternative regularization methods to prevent overfitting. Illustrative and non-limiting examples of regularization methods includes early stopping, including drop outs at least at 1, 2, 3, 4, up to all layers, including L1-L2 regularization on at least 1, 2, 3, 4, up to all layers, including skip connections at least at 1, 2, 3, 4, up to all layers. Regularization can be performed using batch normalization or group normalization.

In some embodiments, the decoder is optimized using any of the following non-limiting optimization procedures: Adam, RMS prop, stochastic gradient descent (SGD) with momentum, SGD with momentum and Nestrop, SGD without momentum, Adagrad, Adadelta, or NAdam. A model can be optimized using any of the follow activation functions: softmax, elu, SeLU, softplus, softsign, ReLU, tan h, sigmoid, hard_sigmoid, exponential, PReLU, and LeaskyReLU, or linear.

In some embodiments, the decoder comprises 3 layers to 100,000 layers. In some embodiments, the decoder comprises 3 layers to 5 layers, 3 layers to 10 layers, 3 layers to 50 layers, 3 layers to 100 layers, 3 layers to 500 layers, 3 layers to 1,000 layers, 3 layers to 5,000 layers, 3 layers to 10,000 layers, 3 layers to 50,000 layers, 3 layers to 100,000 layers, 3 layers to 100,000 layers, 5 layers to 10 layers, 5 layers to 50 layers, 5 layers to 100 layers, 5 layers to 500 layers, 5 layers to 1,000 layers, 5 layers to 5,000 layers, 5 layers to 10,000 layers, 5 layers to 50,000 layers, 5 layers to 100,000 layers, 5 layers to 100,000 layers, 10 layers to 50 layers, 10 layers to 100 layers, 10 layers to 500 layers, 10 layers to 1,000 layers, 10 layers to 5,000 layers, 10 layers to 10,000 layers, 10 layers to 50,000 layers, 10 layers to 100,000 layers, 10 layers to 100,000 layers, 50 layers to 100 layers, 50 layers to 500 layers, 50 layers to 1,000 layers, 50 layers to 5,000 layers, 50 layers to 10,000 layers, 50 layers to 50,000 layers, 50 layers to 100,000 layers, 50 layers to 100,000 layers, 100 layers to 500 layers, 100 layers to 1,000 layers, 100 layers to 5,000 layers, 100 layers to 10,000 layers, 100 layers to 50,000 layers, 100 layers to 100,000 layers, 100 layers to 100,000 layers, 500 layers to 1,000 layers, 500 layers to 5,000 layers, 500 layers to 10,000 layers, 500 layers to 50,000 layers, 500 layers to 100,000 layers, 500 layers to 100,000 layers, 1,000 layers to 5,000 layers, 1,000 layers to 10,000 layers, 1,000 layers to 50,000 layers, 1,000 layers to 100,000 layers, 1,000 layers to 100,000 layers, 5,000 layers to 10,000 layers, 5,000 layers to 50,000 layers, 5,000 layers to 100,000 layers, 5,000 layers to 100,000 layers, 10,000 layers to 50,000 layers, 10,000 layers to 100,000 layers, 10,000 layers to 100,000 layers, 50,000 layers to 100,000 layers, 50,000 layers to 100,000 layers, or 100,000 layers to 100,000 layers. In some embodiments, the decoder comprises 3 layers, 5 layers, 10 layers, 50 layers, 100 layers, 500 layers, 1,000 layers, 5,000 layers, 10,000 layers, 50,000 layers, 100,000 layers, or 100,000 layers. In some embodiments, the decoder comprises at least 3 layers, 5 layers, 10 layers, 50 layers, 100 layers, 500 layers, 1,000 layers, 5,000 layers, 10,000 layers, 50,000 layers, or 100,000 layers. In some embodiments, the decoder comprises at most 5 layers, 10 layers, 50 layers, 100 layers, 500 layers, 1,000 layers, 5,000 layers, 10,000 layers, 50,000 layers, 100,000 layers, or 100,000 layers.

In some embodiments, the decoder is trained to predict the raw amino acid sequence of a protein or polypeptide given an embedding of the sequence. In some embodiments, the decoder is generated at least in part using transfer learning to improve performance. The starting point can be a full first model frozen except the output layer (or one or more additional layers), which is trained on the target protein function or protein feature. The starting point can be the pretrained model, in which the embedding layer, last 2 layers, last 3 layers, or all layers are unfrozen and the rest of the model is frozen during training on the target protein function or protein feature.

In some embodiments, a decoder is trained using a similar procedure to how the encoder is trained. For example, a training set of sequences is obtained, and the trained encoder is used to create embeddings for those sequences. These embeddings represent the input for the decoder, while the output are the original sequences, which the decoder has to predict. In some embodiments, a convolutional neural network is utilized for the decoder that mirrors the architecture of the encoder in reverse. Other types of neural networks can be used, for example, recurrent neural networks (RNNs) such as long short-term memory (LSTM) networks.

The decoder can be trained to minimize the loss, reside-wise categorical cross-entropy, to reconstruct the sequence which maps to a given embedding (also referred to as reconstruction loss). In some embodiments, an additional term is added to the loss, which has been found to provide a substantial improvement to the process. The following notations are used herein:
a. x: a sequence of amino acids
b. y: a measurable property of interest for x, e.g., fluorescence
c. ƒ(x): a function that takes in x to predict y, e.g., a deep neural network
d. enc(x): a submodule of ƒ(x) that produces an embedding (e) of the sequence (x)
e. dec(e): a separate decoder module that takes an embedding (e) and produces a reconstructed sequence (x′)
f. x′: the output of the decoder dec(e), e.g., a reconstructed sequence generated from an embedding (e)

In addition to the reconstruction loss, the reconstructed sequence (x′) is fed back through the original supervised model, f(x′), to produce a predicted value using the decoder's reconstructed sequence (call this y′). The predicted value of the reconstructed sequence (y′) is compared to the predicted value for a given sequence (call this y* and it is computed using f(x)). Similar x and x′ values and/or similar y′ and y* values indicate that the decoder is working effectively. To enforce this, in some embodiments, an additional term is added to the network's loss function using the Kullback-Leibler divergence (KLD). KLD between an arbitrary y′ and y* is represented as:

$$a . \\ KLD ( {{y}^{⋀}}^{′} , {y}^{⋀ *} ) = {{y}^{⋀}}^{′} * log ( {y}^{⋀ *} / {y}^{′} )$$

The loss which incorporates this is represented as:
a. loss=λ_1*CCE+λ_2*KLD(y\hat{ }′,y\hat{ }*), where CCE is the categorical cross-entropy reconstruction loss and λ_1 and λ_2 are tuning parameters.

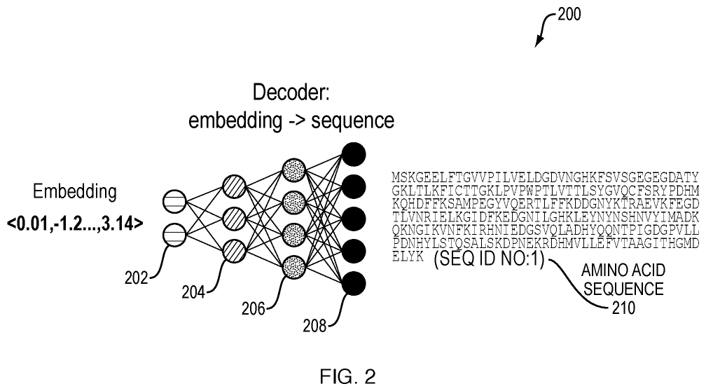

FIG. 2 is a diagram illustrating an example of a decoder as a neural network. The decoder network 200 has four layers of nodes with the first layer 202 corresponding to the embedding layer, which can receive input from the encoder described herein. In this illustrative example, the next two layers 204 and 206 are hidden layers, and the last layer 208 is the final layer that outputs the amino acid sequence that is “decoded” from the embedding.

FIG. 3A is a diagram illustrating an embodiment an overview of the gradient-based design procedure. The encoder 310 can be used to generate a source embedding 304. The source embedding is fed into the decoder 306, which is then turned into a probabilistic sequence (e.g., a distribution of amino acids at each residue). The probabilistic sequence can then be processed by the supervised model 308 comprising the encoder 310 to produce a predicted function value 312. The gradients 314 of function (F) model are taken with respect to the input embedding 304 and are computed by using back-propagation through the supervised model and decoder.

FIG. 3C shows an example of a probabilistic biopolymer sequence 390 produced by a decoder. In this example, the probabilistic biopolymer sequence 390 may be illustrated by a matrix 392. The columns of the matrix 392 represent each of the 20 possible amino acids, and the rows represent the residue position in the protein which has a length L. The first amino acid (row 1) is always a methionine and thus M (column 7) has a probability of 1 and rest of the amino acids has probability 0. The next residue (row 2), as an example, can have a W with 80% probability and a G with 20% probability. To generate a sequence, the maximum likelihood sequence implied by this matrix can be selected, which entails selection of the amino acid with the highest probability at each position. Alternatively, sequences can be randomly generated by sampling each position according to the amino acid probabilities, for example, by randomly picking a W or G at position 2 with 80% vs. 20% probabilities, respectively.

Decoder Validation

In some embodiments, the devices, software, systems, and methods disclosed herein provide decoder validation framework to determine performance of the decoder. An effective decoder is able to predict which sequence maps to a given embedding with very high accuracy. Accordingly, a decoder can be validated by processing the same input (e.g., amino acid sequence) using both a encoder and the encoder-decoder framework described herein. The encoder will generate an output indicative of the desired function and/or property that serves as the reference by which the output of the encoder-decoder framework can be evaluated. As an illustrative example, the encoder and decoder are generated according to the approaches described herein. Next, each protein in training and validation sets are embedded using the encoder. Then, those embeddings are decoded using the decoder. Finally, functional values of the decoded sequence are predicted using the encoder, and compared these predicted values to the values predicted using the original sequence.

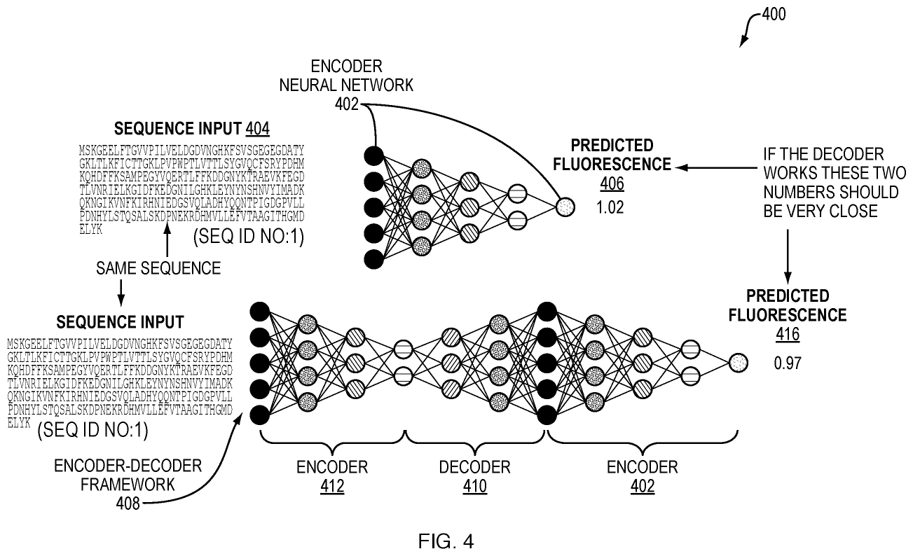

A summary of one embodiment of the decoder validation process 400 is shown in FIG. 4. As shown in FIG. 4, an encoder neural network 402 is shown at the top which receives as input the primary amino acid sequence (e.g., for a green fluorescent protein) and processes the sequence to outputs a prediction 406 of function (e.g., fluorescence intensity). The encoder-decoder framework 408 below shows the encoder network 412 with a penultimate embedding layer that is identical to the encoder neural network 402 except for the missing computation of the prediction 406. The encoder network 412 is connected or linked (or otherwise provides input) to the decoder network 410 to decode the sequence, which is then fed into the encoder network 402 again to arrive at the predicted function 416. Accordingly, when the values of the two predictions 406 and 416 are close, this result provides validation that the decoder 410 is effectively mapping the embedding into a sequence that corresponds to the desired function.

The similarity or correspondence between the predicted values can be computed in any number of ways. In some embodiments, the correlation between the predicted values from the original sequence and the predicted values from the decoded sequence is determined. In some embodiments, the correlation is about 0.7 to about 0.99. In some embodiments, the correlation is about 0.7 to about 0.75, about 0.7 to about 0.8, about 0.7 to about 0.85, about 0.7 to about 0.9, about 0.7 to about 0.95, about 0.7 to about 0.99, about 0.75 to about 0.8, about 0.75 to about 0.85, about 0.75 to about 0.9, about 0.75 to about 0.95, about 0.75 to about 0.99, about 0.8 to about 0.85, about 0.8 to about 0.9, about 0.8 to about 0.95, about 0.8 to about 0.99, about 0.85 to about 0.9, about 0.85 to about 0.95, about 0.85 to about 0.99, about 0.9 to about 0.95, about 0.9 to about 0.99, or about 0.95 to about 0.99. In some embodiments, the correlation is about 0.7, about 0.75, about 0.8, about 0.85, about 0.9, about 0.95, or about 0.99. In some embodiments, the correlation is at least about 0.7, about 0.75, about 0.8, about 0.85, about 0.9, or about 0.95. In some embodiments, the correlation is at most about 0.75, about 0.8, about 0.85, about 0.9, about 0.95, or about 0.99.

Additional performance metrics can be used to validate the systems and methods disclosed herein, for example, positive predictive value (PPV), F1, mean-squared error, area under the receiver operating characteristic (ROC), and area under the precision-recall curve (PRC).

In some embodiments, the methods disclosed herein generate results having a positive predictive value (PPV). In some embodiments, the PPV is 0.7 to 0.99. In some embodiments, the PPV is 0.7 to 0.75, 0.7 to 0.8, 0.7 to 0.85, 0.7 to 0.9, 0.7 to 0.95, 0.7 to 0.99, 0.75 to 0.8, 0.75 to 0.85, 0.75 to 0.9, 0.75 to 0.95, 0.75 to 0.99, 0.8 to 0.85, 0.8 to 0.9, 0.8 to 0.95, 0.8 to 0.99, 0.85 to 0.9, 0.85 to 0.95, 0.85 to 0.99, 0.9 to 0.95, 0.9 to 0.99, or 0.95 to 0.99. In some embodiments, the PPV is 0.7, 0.75, 0.8, 0.85, 0.9, 0.95, or 0.99. In some embodiments, the PPV is at least 0.7, 0.75, 0.8, 0.85, 0.9, or 0.95. In some embodiments, the PPV is at most 0.75, 0.8, 0.85, 0.9, 0.95, or 0.99.

In some embodiments, the methods disclosed herein generate results having an F1 value. In some embodiments, the F1 is 0.5 to 0.95. In some embodiments, the F1 is 0.5 to 0.6, 0.5 to 0.7, 0.5 to 0.75, 0.5 to 0.8, 0.5 to 0.85, 0.5 to 0.9, 0.5 to 0.95, 0.6 to 0.7, 0.6 to 0.75, 0.6 to 0.8, 0.6 to 0.85, 0.6 to 0.9, 0.6 to 0.95, 0.7 to 0.75, 0.7 to 0.8, 0.7 to 0.85, 0.7 to 0.9, 0.7 to 0.95, 0.75 to 0.8, 0.75 to 0.85, 0.75 to 0.9, 0.75 to 0.95, 0.8 to 0.85, 0.8 to 0.9, 0.8 to 0.95, 0.85 to 0.9, 0.85 to 0.95, or 0.9 to 0.95. In some embodiments, the F1 is 0.5, 0.6, 0.7, 0.75, 0.8, 0.85, 0.9, or 0.95. In some embodiments, the F1 is at least 0.5, 0.6, 0.7, 0.75, 0.8, 0.85, or 0.9. In some embodiments, the F1 is at most 0.6, 0.7, 0.75, 0.8, 0.85, 0.9, or 0.95.

In some embodiments, the methods disclosed herein generate results having a mean-squared error. In some embodiments, the mean squared error is 0.01 to 0.3. In some embodiments, the mean squared error is 0.01 to 0.05, 0.01 to 0.1, 0.01 to 0.15, 0.01 to 0.2, 0.01 to 0.25, 0.01 to 0.3, 0.05 to 0.1, 0.05 to 0.15, 0.05 to 0.2, 0.05 to 0.25, 0.05 to 0.3, 0.1 to 0.15, 0.1 to 0.2, 0.1 to 0.25, 0.1 to 0.3, 0.15 to 0.2, 0.15 to 0.25, 0.15 to 0.3, 0.2 to 0.25, 0.2 to 0.3, or 0.25 to 0.3. In some embodiments, the mean squared error is 0.01, 0.05, 0.1, 0.15, 0.2, 0.25, or 0.3. In some embodiments, the mean squared error is at least 0.01, 0.05, 0.1, 0.15, 0.2, or 0.25. In some embodiments, the mean squared error is at most 0.05, 0.1, 0.15, 0.2, 0.25, or 0.3.

In some embodiments, the methods disclosed herein generate results having an area under the ROC. In some embodiments, the area under the ROC 0.7 to 0.95. In some embodiments, the area under the ROC 0.95 to 0.9, 0.95 to 0.85, 0.95 to 0.8, 0.95 to 0.75, 0.95 to 0.7, 0.9 to 0.85, 0.9 to 0.8, 0.9 to 0.75, 0.9 to 0.7, 0.85 to 0.8, 0.85 to 0.75, 0.85 to 0.7, 0.8 to 0.75, 0.8 to 0.7, or 0.75 to 0.7. In some embodiments, the area under the ROC 0.95, 0.9, 0.85, 0.8, 0.75, or 0.7. In some embodiments, the area under the ROC at least 0.95, 0.9, 0.85, 0.8, or 0.75. In some embodiments, the area under the ROC at most 0.9, 0.85, 0.8, 0.75, or 0.7.

In some embodiments, the methods disclosed herein generate results having an area under the PRC. In some embodiments, the area under the PRC 0.7 to 0.95. In some embodiments, the area under the PRC 0.95 to 0.9, 0.95 to 0.85, 0.95 to 0.8, 0.95 to 0.75, 0.95 to 0.7, 0.9 to 0.85, 0.9 to 0.8, 0.9 to 0.75, 0.9 to 0.7, 0.85 to 0.8, 0.85 to 0.75, 0.85 to 0.7, 0.8 to 0.75, 0.8 to 0.7, or 0.75 to 0.7. In some embodiments, the area under the PRC 0.95, 0.9, 0.85, 0.8, 0.75, or 0.7. In some embodiments, the area under the PRC at least 0.95, 0.9, 0.85, 0.8, or 0.75. In some embodiments, the area under the PRC at most 0.9, 0.85, 0.8, 0.75, or 0.7.

Prediction of Polypeptide Sequences

Described herein are devices, software, systems, and methods for evaluating input data such as an initial amino acid sequence (or a nucleic acid sequence that codes for the amino acid sequences) in order to predict one or more novel amino acid sequences corresponding to polypeptides or proteins configured to have specific functions or properties. The extrapolation of specific amino acid sequences (e.g., proteins) capable of performing certain function(s) or having certain properties has long been a goal of molecular biology. Accordingly, the devices, software, systems, and methods described herein leverage the capabilities of artificial intelligence or machine learning techniques for polypeptide or protein analysis to make predictions about sequence information. Machine learning techniques enable the generation of models with increased predictive ability compared to standard non-ML approaches. In some cases, transfer learning is leveraged to enhance predictive accuracy when insufficient data is available to train the model for the desired output. Alternatively, in some cases, transfer learning is not utilized when there is sufficient data to train the model to achieve comparable statistical parameters as a model that incorporates transfer learning.

In some embodiments, input data comprises the primary amino acid sequence for a protein or polypeptide. In some cases, the models are trained using labeled training data sets comprising the primary amino acid sequence. For example, the data set can include amino acid sequences of fluorescent proteins that are labeled based on the degree of fluorescence intensity. Accordingly, a model can be trained on this data set using a machine learning method to generate a prediction of fluorescence intensity for amino acid sequence inputs. In other words, the model can be an encoder such as a deep neural network trained to predict a function based on a primary amino acid sequence input. In some embodiments, the input data comprises information in addition to the primary amino acid sequence such as, for example, surface charge, hydrophobic surface area, measured or predicted solubility, or other relevant information. In some embodiments, the input data comprises multi-dimensional input data including multiple types or categories of data.

In some embodiments, the devices, software, systems, and methods described herein utilize data augmentation to enhance performance of the predictive model(s). Data augmentation entails training using similar but different examples or variations of the training data set. As an example, in image classification, the image data can be augmented by slightly altering the orientation of the image (e.g., slight rotations). In some embodiments, the data inputs (e.g., primary amino acid sequence) are augmented by random mutation and/or biologically informed mutation to the primary amino acid sequence, multiple sequence alignments, contact maps of amino acid interactions, and/or tertiary protein structure. Additional augmentation strategies include the use of known and predicted isoforms from alternatively spliced transcripts. For example, input data can be augmented by including isoforms of alternatively spliced transcripts that correspond to the same function or property. Accordingly, data on isoforms or mutations can allow the identification of those portions or features of the primary sequence that do not significantly impact the predicted function or property. This allows a model to account for information such as, for example, amino acid mutations that enhance, decrease, or do not affect a predicted protein property such as stability. For example, data inputs can comprise sequences with random substituted amino acids at positions that are known not to affect function. This allows the models that are trained on this data to learn that the predicted function is invariant with respect to those particular mutations.

The devices, software, systems, and methods described herein can be used to generate sequence predictions based on one or more of a variety of different functions and/or properties. The predictions can involve protein functions and/or properties (e.g., enzymatic activity, stability, etc.). Amino acid sequences can be predicted or mapped based on protein stability, which can include various metrics such as, for example, thermostability, oxidative stability, or serum stability. In some embodiments, an encoder is configured to incorporate information relating to one or more structural features such as, for example, secondary structure, tertiary protein structure, quaternary structure, or any combination thereof. Secondary structure can include a designation of whether an amino acid or a sequence of amino acids in a polypeptide is predicted to have an alpha helical structure, a beta sheet structure, or a disordered or loop structure. Tertiary structure can include the location or positioning of amino acids or portions of the polypeptide in three-dimensional space. Quaternary structure can include the location or positioning of multiple polypeptides forming a single protein. In some embodiments, a prediction comprises a sequence based on one or more functions. Polypeptide or protein functions can belong to various categories including metabolic reactions, DNA replication, providing structure, transportation, antigen recognition, intracellular or extracellular signaling, and other functional categories. In some embodiments, a prediction comprises an enzymatic function such as, for example, catalytic efficiency (e.g., specificity constant k_{cat}/K_{M}) or catalytic specificity.

In some embodiments, a sequence prediction is based on an enzymatic function for a protein or polypeptide. In some embodiments, a protein function is an enzymatic function. Enzymes can perform various enzymatic reactions and can be categorized as transferases (e.g., transfers functional groups from one molecule to another), oxidoreductases (e.g., catalyzes oxidation-reduction reactions), hydrolases (e.g., cleaves chemical bonds via hydrolysis), lyases (e.g., generate a double bond), ligases (e.g., joining two molecules via a covalent bond), and isomerases (e.g., catalyzes structural changes within a molecule from one isomer to another). In some embodiments, hydrolases include proteases such as serine proteases, threonine proteases, cysteine proteases, metalloproteases, asparagine peptide lyases, glutamic proteases, and aspartic proteases. Serine proteases have various physiological roles such as in blood coagulation, wound healing, digestion, immune responses and tumor invasion and metastasis. Examples of serine proteases include chymotrypsin, trypsin, elastase, Factor 10, Factor 11, Thrombin, Plasmin, C1r, C1s, and C3 convertases. Threonine proteases include a family of proteases that have a threonine within the active catalytic site. Examples of threonine proteases include subunits of the proteasome. The proteasome is a barrel-shaped protein complex made up of alpha and beta subunits. The catalytically active beta subunit can include a conserved N-terminal threonine at each active site for catalysis. Cysteine proteases have a catalytic mechanism that utilizes a cysteine sulfhydryl group. Examples of cysteine proteases include papain, cathepsin, caspases, and calpains. Aspartic proteases have two aspartate residues that participate in acid/base catalysis at the active site. Examples of aspartatic proteases include the digestive enzyme pepsin, some lysosomal proteases, and renin. Metalloproteases include the digestive enzymes carboxypeptidases, matrix metalloproteases (MMPs) which play roles in extracellular matrix remodeling and cell signaling, ADAMs (a disintegrin and metalloprotease domain), and lysosomal proteases. Other non-limiting examples of enzymes include proteases, nucleases, DNA ligases, polymerases, cellulases, liginases, amylases, lipases, pectinases, xylanases, lignin peroxidases, decarboxylases, mannanases, dehydrogenases, and other polypeptide-based enzymes.

In some embodiments, enzymatic reactions include post-translational modifications of target molecules. Examples of post-translational modifications include acetylation, amidation, formylation, glycosylation, hydroxylation, methylation, myristoylation, phosphorylation, deamidation, prenylation (e.g., farnesylation, geranylation, etc.), ubiquitylation, ribosylation and sulphation. Phosphorylation can occur on an amino acid such as tyrosine, serine, threonine, or histidine.

In some embodiments, the protein function is luminescence which is light emission without requiring the application of heat. In some embodiments, the protein function is chemiluminescence such as bioluminescence. For example, a chemiluminescent enzyme such as luciferin can act on a substrate (luciferin) to catalyze the oxidation of the substrate, thereby releasing light. In some embodiments, the protein function is fluorescence in which the fluorescent protein or peptide absorbs light of certain wavelength(s) and emits light at different wavelength(s). Examples of fluorescent proteins include green fluorescent protein (GFP) or derivatives of GFP such as EBFP, EBFP2, Azurite, mKalamal, ECFP, Cerulean, CyPet, YFP, Citrine, Venus, or YPet. Some proteins such as GFP are naturally fluorescent. Examples of fluorescent proteins include EGFP, blue fluorescent protein (EBFP, EBFP2, Azurite, mKalamal), cyan fluorescent protein (ECFP, Cerulean, CyPet), yellow fluorescent protein (YFP, Citrine, Venus, YPet), redox sensitive GFP (roGFP), and monomeric GFP.

In some embodiments, the protein function comprises an enzymatic function, binding (e.g., DNA/RNA binding, protein binding, etc.), immune function (e.g., antibody), contraction (e.g., actin, myosin), and other functions. In some embodiments, the output comprises a primary sequence associated with the protein function such as, for example, kinetics of enzymatic function or binding. As an example, such outputs can be obtained by optimizing a composite function that incorporates desired metrics such as any of affinity, specificity, or reaction rate.

In some embodiments, the systems and methods disclosed herein generate biopolymer sequences corresponding to a function or property. In some cases, the biopolymer sequence is a nucleic acid. In some cases, the biopolymer sequence is a polypeptide. Examples of specific biopolymer sequences include fluorescent proteins such as GFP and enzymes such as beta-lactamase. In one instance, a reference GFP sequence such as avGFP is defined by a 238 amino acid long polypeptide having the following sequence:

(SEQ ID NO: 1)
MSKGEELFTGVVPILVELDGDVNGHKFSVSGEGEGDATYGKLTLKFICT
TGKLPVPWPTLVTTFSYGVQCFSRYPDHMKQHDFFKSAMPEGYVQERTI
FFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLP
DNHYLSTQSALSKDPNEKRDHMVLLEFVTAAGITHGMDELYK.

A GFP sequence designed using gradient-based design can comprise a sequence that has less than 100% sequence identity to the reference GFP sequence. In some cases, the GBD-optimized GFP sequence has a sequence identity with respect to SEQ ID NO: 1 of 80% to 99%. In some cases, the GBD-optimized GFP sequence has a sequence identity with respect to SEQ ID NO: 1 of 80% to 85%, 80% to 90%, 80% to 95%, 80% to 96%, 80% to 97%, 80% to 98%, 80% to 99%, 85% to 90%, 85% to 95%, 85% to 96%, 85% to 97%, 85% to 98%, 85% to 99%, 90% to 95%, 90% to 96%, 90% to 97%, 90% to 98%, 90% to 99%, 95% to 96%, 95% to 97%, 95% to 98%, 95% to 99%, 96% to 97%, 96% to 98%, 96% to 99%, 97% to 98%, 97% to 99%, or 98% to 99%. In some cases, the GBD-optimized GFP sequence has a sequence identity with respect to SEQ ID NO: 1 of 80%, 85%, 90%, 95%, 96%, 97%, 98%, or 99%. In some cases, the GBD-optimized GFP sequence has a sequence identity with respect to SEQ ID NO: 1 of at least 80%, 85%, 90%, 95%, 96%, 97%, or 98%. In some cases, the GBD-optimized GFP sequence has a sequence identity with respect to SEQ ID NO: 1 of at most 85%, 90%, 95%, 96%, 97%, 98%, or 99%. In some cases, the GBD-optimized GFP sequence has less than 45 (e.g., less than: 40, 35, 30, 25, 20, 15, or 10) amino acid substitutions, relative to SEQ ID NO:1. In some cases, the GBD-optimized GFP sequence comprises at least one, two, three, four, five, six, or seven point mutations relative to the reference GFP sequence. The GBD-optimized GFP sequence can be defined by one or more mutations selected from Y39C, F64L, V68M, D129G, V163A, K166R, and G191V, including combinations of the foregoing, e.g., including 1, 2, 3, 4, 5, 6, or all 7 mutations. In some cases, the GBD-optimized GFP sequence does not include a S65T mutation. The GBD-optimized GFP sequences provided by the invention, in some embodiments, include an N-terminal methionine, while in other embodiments the sequences do not include an N-terminal methionine.

In some embodiments, disclosed herein are nucleic acid sequences encoding GBD-optimized polypeptide sequences such as GFP and/or beta-lactamase. Also disclosed herein are vectors comprising the nucleic acid sequence, for example, a prokaryotic and/or eukaryotic expression vector. The expression vectors may be constitutively active or have inducible expression (e.g., tetracycline-inducible promoters). For example, CMV promoters are constitutively active but can also be regulated using Tet Operator elements that allow induction of expression in the presence of tetracycline/doxycycline.

The polypeptides and nucleic acid sequences encoding the same can be used in various imaging techniques. For example, fluorescence microscopy, cell activated cell sorting (FACS), flow cytometry, and other fluorescence-imaging based techniques can utilize the fluorescent proteins of the present disclosure. A GBD-optimized GFP protein can provide greater brightness than standard reference GFP proteins. In some cases, the GBD-optimized GFP protein has a fluorescence brightness that is greater than 2, 3, 4, 5, 6, 7, 8, 9, 10, 15, 20, 25, 30, 35, 40, 45, or 50 fold or more compared to the brightness of a non-optimized GFP sequence (e.g., avGFP).

In some embodiments, the machine learning method(s) described herein comprise supervised machine learning. Supervised machine learning includes classification and regression. In some embodiments, the machine learning method(s) comprise unsupervised machine learning. Unsupervised machine learning includes clustering, autoencoding, variational autoencoding, protein language model (e.g., wherein the model predicts the next amino acid in a sequence when given access to the previous amino acids), and association rules mining.

### Machine Learning

Described herein are devices, software, systems, and methods that apply one or more methods for analyzing input data to generate a sequence mapped to one or more protein or polypeptide properties or functions. In some embodiments, the methods utilize statistical modeling to generate predictions or estimates about protein or polypeptide function(s) or properties. In some embodiments, methods are used to embed primary sequences such as amino acid sequences into an embedding space, optimize the embedded sequence with respect to a desired function or property, and to process the optimized embedding to generate a sequence predicted to have the function or property. In some embodiments, an encoder-decoder framework is utilized in which two models are combined to allow an initial sequence to be embedded using a first model, and then for an optimized embedding to be mapped onto a sequence using a second model.

In some embodiments, a method utilizes a predictive model such as a neural network, a decision tree, a support vector machine, or other applicable model. Using the training data, a method is able to form a classifier for generating a classification or prediction according to relevant features. The features selected for classification can be classified using a variety of methods. In some embodiments, the trained method comprises a machine learning method.

In some embodiments, the machine learning method uses a support vector machine (SVM), a Naïve Bayes classification, a random forest, or an artificial neural network. Machine learning techniques include bagging procedures, boosting procedures, random forest methods, and combinations thereof. In some embodiments, the predictive model is a deep neural network. In some embodiments, the predictive model is a deep convolutional neural network.

In some embodiments, a machine learning method uses a supervised learning approach. In supervised learning, the method generates a function from labeled training data. Each training example is a pair consisting of an input object and a desired output value. In some embodiments, an optimal scenario allows for the method to correctly determine the class labels for unseen instances. In some embodiments, a supervised learning method requires the user to determine one or more control parameters. These parameters are optionally adjusted by optimizing performance on a subset, called a validation set, of the training set. After parameter adjustment and learning, the performance of the resulting function is optionally measured on a test set that is separate from the training set. Regression methods are commonly used in supervised learning. Accordingly, supervised learning allows for a model or classifier to be generated or trained with training data in which the expected output is known in advance such as in calculating a protein function when the primary amino acid sequence is known.

In some embodiments, a machine learning method uses an unsupervised learning approach. In unsupervised learning, the method generates a function to describe hidden structures from unlabeled data (e.g., a classification or categorization is not included in the observations). Since the examples given to the learner are unlabeled, there is no evaluation of the accuracy of the structure that is output by the relevant method. Approaches to unsupervised learning include: clustering, anomaly detection, and approaches based on neural networks including autoencoders and variational autoencoders.

In some embodiments, the machine learning method utilizes multi-class learning. Multi-task learning (MTL) is an area of machine learning in which more than one learning task is solved simultaneously in a manner that takes advantage of commonalities and differences across the multiple tasks. Advantages of this approach can include improved learning efficiency and prediction accuracy for the specific predictive models in comparison to training those models separately. Regularization to prevent overfitting can be provided by requiring a method to perform well on a related task. This approach can be better than regularization that applies an equal penalty to all complexity. Multi-class learning can be especially useful when applied to tasks or predictions that share significant commonalities and/or are under-sampled. In some embodiments, multi-class learning is effective for tasks that do not share significant commonalities (e.g., unrelated tasks or classifications). In some embodiments, multi-class learning is used in combination with transfer learning.

In some embodiments, a machine learning method learns in batches based on the training dataset and other inputs for that batch. In other embodiments, the machine learning method performs additional learning where the weights and error calculations are updated, for example, using new or updated training data. In some embodiments, the machine learning method updates the prediction model based on new or updated data. For example, a machine learning method can be applied to new or updated data to be re-trained or optimized to generate a new prediction model. In some embodiments, a machine learning method or model is re-trained periodically as additional data becomes available.

In some embodiments, the classifier or trained method of the present disclosure comprises one feature space. In some cases, the classifier comprises two or more feature spaces. In some embodiments, the two or more feature spaces are distinct from one another. In some embodiments, the accuracy of the classification or prediction is improved by combining two or more feature spaces in a classifier instead of using a single feature space. The attributes generally make up the input features of the feature space and are labeled to indicate the classification of each case for the given set of input features corresponding to that case.

In some embodiments, one or more sets of training data are used to train a model using a machine learning method. In some embodiments, the methods described herein comprise training a model using a training data set. In some embodiments, the model is trained using a training data set comprising a plurality of amino acid sequences. In some embodiments, the training data set comprises at least 1, 2, 3, 4, 5, 6, 7, 8, 9, 10, 15, 20, 25, 30, 35, 40, 45, 50, 55, 56, 57, 58 million protein amino acid sequences. In some embodiments, the training data set comprises at least 10, 20, 30, 40, 50, 60, 70, 80, 90, 100, 150, 200, 250, 300, 350, 400, 450, 500, 600, 700, 800, 900, or 1000 thousand or more amino acid sequences. In some embodiments, the training data set comprises at least 50, 100, 200, 300, 400, 500, 600, 700, 800, 900, 1000, 2000, 3000, 4000, 5000, 6000, 7000, 8000, 9000, or 10000 or more annotations. Although exemplar embodiments of the present disclosure include machine learning methods that use deep neural networks, various types of methods are contemplated. In some embodiments, the method utilizes a predictive model such as a neural network, a decision tree, a support vector machine, or other applicable model. In some embodiments, the machine learning method is selected from the group consisting of a supervised, semi-supervised and unsupervised learning, such as, for example, a support vector machine (SVM), a Naïve Bayes classification, a random forest, an artificial neural network, a decision tree, a K-means, learning vector quantization (LVQ), self-organizing map (SOM), graphical model, regression method (e.g., linear, logistic, multivariate, association rule learning, deep learning, dimensionality reduction and ensemble selection methods. In some embodiments, the machine learning method is selected from the group consisting of: a support vector machine (SVM), a Naïve Bayes classification, a random forest, and an artificial neural network. Machine learning techniques include bagging procedures, boosting procedures, random forest methods, and combinations thereof. Illustrative methods for analyzing the data include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis.

The various models described herein, including supervised and unsupervised models, can have alternative regularization methods, including early stopping, including drop outs at 1, 2, 3, 4, up to all layers, including L1-L2 regularization on 1, 2, 3, 4, up to all layers, including skip connections at 1, 2, 3, 4, up to all layers. For both the first model and the second model, regularization can be performed using batch normalization or group normalization. L1 regularization (also known as the LASSO) controls how long the L1 norm of the weight vector is allowed to be, whereas L2 controls how large the L2 norm can be. Skip connections can be obtained from the Resnet architecture.

The various models trained using machine learning described herein can be optimized using any of the following optimization procedures: Adam, RMS prop, stochastic gradient descent (SGD) with momentum, SGD with momentum and Nestrov accelerated gradient, SGD without momentum, Adagrad, Adadelta, or NAdam. A model can be optimized using any of the follow activation functions: softmax, elu, SeLU, softplus, softsign, ReLU, tan h, sigmoid, hard_sigmoid, exponential, PReLU, and LeaskyReLU, or linear. A loss function can be used to measure the performance of a model. The loss can be understood as the cost of the inaccuracy of the prediction. For example, a cross-entropy loss function measures the performance of a classification model having an output that is a probability value between 0 and 1 (e.g., 0 being no antibiotic resistance and 1 being complete antibiotic resistance). This loss value increases as the predicted probability diverges from the actual value.

In some embodiments, the methods described herein comprise “reweighting” the loss function that the optimizers listed above attempt to minimize, so that approximately equal weight is placed on both positive and negative examples. For example, one of the 180,000 outputs predicts the probability that a given protein is a membrane protein. Since a protein can only be a membrane protein or not a membrane protein, this is binary classification task, and the traditional loss function for a binary classification task is “binary cross-entropy”: loss(p,y)=−y*log(p)−(1−y)*log(1−p), where p is the probability of being a membrane protein according to the network and y is the “label” which is 1 if the protein is a membrane protein and 0 if it is not. A problem may arise if there are far more examples of y=0 because the network will likely learn the pathological rule of always predicting extremely low probabilities for this annotation because it is rarely penalized for always predicting y=0. To get around this, in some embodiments, the loss function is modified to the following: loss(p,y)=−w1*y*log(p)−w0*(1−y)*log(1−p), where w1 is the weight for the positive class and w0 is the weight for the negative class. This approach assumes w0=1 and ]w1=1/√((1−f0)/f1), where f0 is the frequency of negative examples and f1 is the frequency of positive examples. This weighting scheme “upweights” the positive examples which are rare, and “downweights” the negative examples which are more common. Thus, the methods disclosed herein can comprise incorporating a weighting scheme providing an upweight and/or downweight into a loss function to account for uneven distribution of the negative and positive examples.

In some embodiments, a trained model such as a neural network comprises 10 layers to 1,000,000 layers. In some embodiments, the neural network comprises 10 layers to 50 layers, 10 layers to 100 layers, 10 layers to 200 layers, 10 layers to 500 layers, 10 layers to 1,000 layers, 10 layers to 5,000 layers, 10 layers to 10,000 layers, 10 layers to 50,000 layers, 10 layers to 100,000 layers, 10 layers to 500,000 layers, 10 layers to 1,000,000 layers, 50 layers to 100 layers, 50 layers to 200 layers, 50 layers to 500 layers, 50 layers to 1,000 layers, 50 layers to 5,000 layers, 50 layers to 10,000 layers, 50 layers to 50,000 layers, 50 layers to 100,000 layers, 50 layers to 500,000 layers, 50 layers to 1,000,000 layers, 100 layers to 200 layers, 100 layers to 500 layers, 100 layers to 1,000 layers, 100 layers to 5,000 layers, 100 layers to 10,000 layers, 100 layers to 50,000 layers, 100 layers to 100,000 layers, 100 layers to 500,000 layers, 100 layers to 1,000,000 layers, 200 layers to 500 layers, 200 layers to 1,000 layers, 200 layers to 5,000 layers, 200 layers to 10,000 layers, 200 layers to 50,000 layers, 200 layers to 100,000 layers, 200 layers to 500,000 layers, 200 layers to 1,000,000 layers, 500 layers to 1,000 layers, 500 layers to 5,000 layers, 500 layers to 10,000 layers, 500 layers to 50,000 layers, 500 layers to 100,000 layers, 500 layers to 500,000 layers, 500 layers to 1,000,000 layers, 1,000 layers to 5,000 layers, 1,000 layers to 10,000 layers, 1,000 layers to 50,000 layers, 1,000 layers to 100,000 layers, 1,000 layers to 500,000 layers, 1,000 layers to 1,000,000 layers, 5,000 layers to 10,000 layers, 5,000 layers to 50,000 layers, 5,000 layers to 100,000 layers, 5,000 layers to 500,000 layers, 5,000 layers to 1,000,000 layers, 10,000 layers to 50,000 layers, 10,000 layers to 100,000 layers, 10,000 layers to 500,000 layers, 10,000 layers to 1,000,000 layers, 50,000 layers to 100,000 layers, 50,000 layers to 500,000 layers, 50,000 layers to 1,000,000 layers, 100,000 layers to 500,000 layers, 100,000 layers to 1,000,000 layers, or 500,000 layers to 1,000,000 layers. In some embodiments, the neural network comprises 10 layers, 50 layers, 100 layers, 200 layers, 500 layers, 1,000 layers, 5,000 layers, 10,000 layers, 50,000 layers, 100,000 layers, 500,000 layers, or 1,000,000 layers. In some embodiments, the neural network comprises at least 10 layers, 50 layers, 100 layers, 200 layers, 500 layers, 1,000 layers, 5,000 layers, 10,000 layers, 50,000 layers, 100,000 layers, or 500,000 layers. In some embodiments, the neural network comprises at most 50 layers, 100 layers, 200 layers, 500 layers, 1,000 layers, 5,000 layers, 10,000 layers, 50,000 layers, 100,000 layers, 500,000 layers, or 1,000,000 layers.

In some embodiments, a machine learning method comprises a trained model or classifier that is tested using data that was not used for training to evaluate its predictive ability. In some embodiments, the predictive ability of the trained model or classifier is evaluated using one or more performance metrics. These performance metrics include classification accuracy, specificity, sensitivity, positive predictive value, negative predictive value, measured area under the receiver operator curve (AUROC), mean squared error, false discover rate, and Pearson correlation between the predicted and actual values which are determined for a model by testing it against a set of independent cases. In some instances, a method has an AUROC of at least about 60%, 65%, 70%, 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, a method has an accuracy of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, a method has a specificity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, a method has a sensitivity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, a method has a positive predictive value of at least about 75%, 800%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances a method has a negative predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein.

### Transfer Learning

Described herein are devices, software, systems, and methods for generating a protein or polypeptide sequence based on one or more desired properties or functions. In some embodiments, transfer learning is used to enhance predictive accuracy. Transfer learning is a machine learning technique where a model developed for one task can be reused as the starting point for a model on a second task. Transfer learning can be used to boost predictive accuracy on a task where there is limited data by having the model learn a on a related task where data is abundant. The transfer learning methods described in PCT Application No. PCT/US2020/01751762/804,036 are herein incorporated by reference. Accordingly, described herein are methods for learning general, functional features of proteins from a large data set of sequenced proteins and using it as a starting point for a model to predict any specific protein function, property, or feature. Thus, generation of an encoder can include transfer learning so as to improve performance of the encoder in processing an input sequence into an embedding. An improved embedding can therefore enhance the performance of the overall encoder-decoder framework. The present disclosure recognizes the surprising discovery that the information encoded in all sequenced proteins by a first predictive model can be transferred to design specific protein functions of interest using a second predictive model. In some embodiments, the predictive models are neural networks such as, for example, deep convolutional neural networks.

The present disclosure can be implemented via one or more embodiments to achieve one or more of the following advantages. In some embodiments, a model trained with transfer learning exhibits improvements from a resource consumption standpoint such as exhibiting a small memory footprint, low latency, or low computational cost. This advantage cannot be understated in complex analyses that can require tremendous computing power. In some cases, the use of transfer learning is necessary to train sufficiently accurate models within a reasonable period of time (e.g., days instead of weeks). In some embodiments, the model trained using transfer learning provides a high accuracy compared to a model not trained using transfer learning. In some embodiments, the use of a deep neural network and/or transfer learning in a system for predicting polypeptide sequence, structure, property, and/or function increases computational efficiency compared to other methods or models that do not use transfer learning.

In some embodiments, a first system is provided comprising a neural net embedder or encoder. In some embodiments, the neural net embedder comprises one or more embedding layers. In some embodiments, the input to the neural network comprises a protein sequence represented as a “one-hot” vector that encodes the sequence of amino acids as a matrix. For example, within the matrix, each row can be configured to contain exactly 1 non-zero entry which corresponds to the amino acid present at that residue. In some embodiments, the first system comprises a neural net predictor. In some embodiments, the predictor comprises one or more output layers for generating a prediction or output based on the input. In some embodiments, the first system is pretrained using a first training data set to provide a pretrained neural net embedder. With transfer learning, the pretrained first system or a portion thereof can be transferred to form part of a second system. The one or more layers of the neural net embedder can be frozen when used in the second system. In some embodiments, the second system comprises the neural net embedder or a portion thereof from the first system. In some embodiments, the second system comprises a neural net embedder and a neural net predictor. The neural net predictor can include one or more output layers for generating a final output or prediction. The second system can be trained using a second training data set that is labeled according to the protein function or property of interest. As used herein, an embedder and a predictor can refer to components of a predictive model such as neural net trained using machine learning. Within the encoder-decoder framework disclosed herein, the embedding layer can be processed for optimization and subsequent “decoding” into an updated or optimized sequence with respect to one or more functions.

In some embodiments, transfer learning is used to train a first model, at least part of which is used to form a portion of a second model. The input data to the first model can comprise a large data repository of known natural and synthetic proteins, regardless of function or other properties. The input data can include any combination of the following: primary amino acid sequence, secondary structure sequences, contact maps of amino acid interactions, primary amino acid sequence as a function of amino acid physicochemical properties, and/or tertiary protein structures. Although these specific examples are provided herein, any additional information relating to the protein or polypeptide is contemplated. In some embodiments, the input data is embedded. For example, the input data can be represented as a multidimensional tensor of binary 1-hot encodings of sequences, real-values (e.g., in the case of physicochemical properties or 3-dimensional atomic positions from tertiary structure), adjacency matrices of pairwise interactions, or using a direct embedding of the data (e.g., character embeddings of the primary amino acid sequence). A first system can comprise a convolutional neural network architecture with an embedding vector and linear model that is trained using UniProt amino acid sequences and ˜70,000 annotations (e.g., sequence labels). During the transfer learning process, the embedding vector and convolutional neural network portion of the first system or model is transferred to form the core of a second system or model that now incorporates a new linear model configured to predict a protein property or function. This second system is trained using a second training data set based on the desired sequence labels corresponding to the protein property or function. Once training is finished, the second system can be assessed against a validation data set and/or a test data set (e.g., data not used in training).

In some embodiments, the data inputs to the first model and/or the second model are augmented by additional data such as random mutation and/or biologically informed mutation to the primary amino acid sequence, contact maps of amino acid interactions, and/or tertiary protein structure. Additional augmentation strategies include the use of known and predicted isoforms from alternatively spliced transcripts. In some embodiments, different types of inputs (e.g., amino acid sequence, contact maps, etc.) are processed by different portions of one or more models. After the initial processing steps, the information from multiple data sources can be combined at a layer in the network. For example, a network can comprise a sequence encoder, a contact map encoder, and other encoders configured to receive and/or process various types of data inputs. In some embodiments, the data is turned into an embedding within one or more layers in the network.

The labels for the data inputs to the first model can be drawn from one or more public protein sequence annotations resources such as, for example: Gene Ontology (GO), Pfam domains, SUPFAM domains, Enzyme Commission (EC) numbers, taxonomy, extremophile designation, keywords, ortholog group assignments including OrthoDB and KEGG Ortholog. In addition, labels can be assigned based on known structural or fold classifications designated by databases such as SCOP, FSSP, or CATH, including all-α, all-β, α+β, α/β, membrane, intrinsically disordered, coiled coil, small, or designed proteins. For proteins for which the structure is known, quantitative global characteristics such as total surface charge, hydrophobic surface area, measured or predicted solubility, or other numeric quantities can be used as additional labels fit by a predictive model such as a multi-task model. Although these inputs are described in the context of transfer learning, the application of these inputs for non-transfer learning approaches is also contemplated. In some embodiments, the first model comprises an annotation layer that is stripped away to leave the core network composed of the encoder. The annotation layer can include multiple independent layers, each corresponding to a particular annotation such as, for example, primary amino acid sequence, GO, Pfam, Interpro, SUPFAM, KO, OrthoDB, and keywords. In some embodiments, the annotation layer comprises at least 1, 2, 3, 4, 5, 6, 7, 8, 9, 10, 15, 20, 25, 30, 40, 50, 60, 70, 80, 90, 100, 1000, 5000, 10000, 50000, 100000, or 150000 or more independent layers. In some embodiments, the annotation layer comprises 180000 independent layers. In some embodiments, a model is trained using at least 1, 2, 3, 4, 5, 6, 7, 8, 9, 10, 15, 20, 25, 30, 40, 50, 60, 70, 80, 90, 100, 1000, 5000, 10000, 50000, 100000, or 150000 or more annotations. In some embodiments, a model is trained using about 180000 annotations. In some embodiments, the model is trained with multiple annotations across a plurality of functional representations (e.g., one or more of GO, Pfam, keywords, Kegg Ontology, Interpro, SUPFAM, and OrthoDB). Amino acid sequence and annotation information can be obtained from various databases such as UniProt.

In some embodiments, the first model and the second model comprise a neural network architecture. The first model and the second model can be a supervised model using a convolutional architecture in the form of a 1D convolution (e.g., primary amino acid sequence), a 2D convolution (e.g., contact maps of amino acid interactions), or a 3D convolution (e.g., tertiary protein structures). The convolutional architecture can be one of the following described architectures: VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, a single model approach (e.g., non-transfer learning) is contemplated that utilizes any of the architectures described herein.

The first model can also be an unsupervised model using either a generative adversarial network (GAN), recurrent neural network, or a variational autoencoder (VAE). If a GAN, the first model can be a conditional GAN, deep convolutional GAN, StackGAN, infoGAN, Wasserstein GAN, Discover Cross-Domain Relations with Generative Adversarial Networks (Disco GANS). In the case of a recurrent neural network, the first model can be a Bi-LSTM/LSTM, a Bi-GRU/GRU, or a transformer network. In some embodiments, a single model approach (e.g., non-transfer learning) is contemplated that utilizes any of the architectures described herein for generating the encoder and/or decoder. In some embodiments, a GAN is DCGAN, CGAN, SGAN/progressive GAN, SAGAN, LSGAN, WGAN, EBGAN, BEGAN, or infoGAN. A recurrent neural network (RNN) is a variant of a tradition neural network built for sequential data. LSTM refers to long short term memory, which is a type of neuron in an RNN with a memory that allows it to model sequential or temporal dependencies in data. GRU refers to gated recurrent unit, which is a variant of the LSTM which attempts to address some the LSTMs shortcomings. Bi-LSTM/Bi-GRU refers to “bidirectional” variants of LSTM and GRU. Typically LSTMs and GRUs process sequential in the “forward” direction, but bi-directional versions learn in the “backward” direction as well. LSTM enables the preservation of information from data inputs that have already passed through it using the hidden state. Unidirectional LSTM only preserves information of the past because it has only seen inputs from the past. By contrast, bidirectional LSTM runs the data inputs in both directions from the past to the future and vice versa. Accordingly, the bidirectional LSTM that runs forwards and backwards preserves information from the future and the past.

The second model can use the first model as a starting point for training. The starting point can be the full first model frozen except the output layer, which is trained on the target protein function or protein property. The starting point can be the first model where the embedding layer, last 2 layers, last 3 layers, or all layers are unfrozen and the rest of the model is frozen during training on the target protein function or protein property. The starting point can be the first model where the embedding layer is removed and 1, 2, 3, or more layers are added and trained on the target protein function or protein property. In some embodiments, the number of frozen layers is 1 to 10. In some embodiments, the number of frozen layers is 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10. In some embodiments, the number of frozen layers is 1, 2, 3, 4, 5, 6, 7, 8, 9, or 10. In some embodiments, the number of frozen layers is at least 1, 2, 3, 4, 5, 6, 7, 8, or 9. In some embodiments, the number of frozen layers is at most 2, 3, 4, 5, 6, 7, 8, 9, or 10. In some embodiments, no layers are frozen during transfer learning. In some embodiments, the number of layers that are frozen in the first model is determined at least partly based on the number of samples available for training the second model. The present disclosure recognizes that freezing layer(s) or increasing the number of frozen layers can enhance the predictive performance of the second model. This effect can be accentuated in the case of low sample size for training the second model. In some embodiments, all the layers from the first model are frozen when the second model has no more than 200, 190, 180, 170, 160, 150, 140, 130, 120, 110, 100, 90, 80, 70, 60, 50, 40, or 30 samples in a training set. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or at least 100 layers in the first model are frozen for transfer to the second model when the number of samples for training the second model is no more than 200, 190, 180, 170, 160, 150, 140, 130, 120, 110, 100, 90, 80, 70, 60, 50, 40, or 30 samples in a training set.

The first and the second model can have 10-100 layers, 100-500 layers, 500-1000 layers, 1000-10000 layers, or up to 1000000 layers. In some embodiments, the first and/or second model comprises 10 layers to 1,000,000 layers. In some embodiments, the first and/or second model comprises 10 layers to 50 layers, 10 layers to 100 layers, 10 layers to 200 layers, 10 layers to 500 layers, 10 layers to 1,000 layers, 10 layers to 5,000 layers, 10 layers to 10,000 layers, 10 layers to 50,000 layers, 10 layers to 100,000 layers, 10 layers to 500,000 layers, 10 layers to 1,000,000 layers, 50 layers to 100 layers, 50 layers to 200 layers, 50 layers to 500 layers, 50 layers to 1,000 layers, 50 layers to 5,000 layers, 50 layers to 10,000 layers, 50 layers to 50,000 layers, 50 layers to 100,000 layers, 50 layers to 500,000 layers, 50 layers to 1,000,000 layers, 100 layers to 200 layers, 100 layers to 500 layers, 100 layers to 1,000 layers, 100 layers to 5,000 layers, 100 layers to 10,000 layers, 100 layers to 50,000 layers, 100 layers to 100,000 layers, 100 layers to 500,000 layers, 100 layers to 1,000,000 layers, 200 layers to 500 layers, 200 layers to 1,000 layers, 200 layers to 5,000 layers, 200 layers to 10,000 layers, 200 layers to 50,000 layers, 200 layers to 100,000 layers, 200 layers to 500,000 layers, 200 layers to 1,000,000 layers, 500 layers to 1,000 layers, 500 layers to 5,000 layers, 500 layers to 10,000 layers, 500 layers to 50,000 layers, 500 layers to 100,000 layers, 500 layers to 500,000 layers, 500 layers to 1,000,000 layers, 1,000 layers to 5,000 layers, 1,000 layers to 10,000 layers, 1,000 layers to 50,000 layers, 1,000 layers to 100,000 layers, 1,000 layers to 500,000 layers, 1,000 layers to 1,000,000 layers, 5,000 layers to 10,000 layers, 5,000 layers to 50,000 layers, 5,000 layers to 100,000 layers, 5,000 layers to 500,000 layers, 5,000 layers to 1,000,000 layers, 10,000 layers to 50,000 layers, 10,000 layers to 100,000 layers, 10,000 layers to 500,000 layers, 10,000 layers to 1,000,000 layers, 50,000 layers to 100,000 layers, 50,000 layers to 500,000 layers, 50,000 layers to 1,000,000 layers, 100,000 layers to 500,000 layers, 100,000 layers to 1,000,000 layers, or 500,000 layers to 1,000,000 layers. In some embodiments, the first and/or second model comprises 10 layers, 50 layers, 100 layers, 200 layers, 500 layers, 1,000 layers, 5,000 layers, 10,000 layers, 50,000 layers, 100,000 layers, 500,000 layers, or 1,000,000 layers. In some embodiments, the first and/or second model comprises at least 10 layers, 50 layers, 100 layers, 200 layers, 500 layers, 1,000 layers, 5,000 layers, 10,000 layers, 50,000 layers, 100,000 layers, or 500,000 layers. In some embodiments, the first and/or second model comprises at most 50 layers, 100 layers, 200 layers, 500 layers, 1,000 layers, 5,000 layers, 10,000 layers, 50,000 layers, 100,000 layers, 500,000 layers, or 1,000,000 layers.

In some embodiments, described herein is a first system comprising a neural net embedder and optionally a neural net predictor. In some embodiments, a second system comprises a neural net embedder and a neural net predictor. In some embodiments, the embedder comprises 10 layers to 200 layers. In some embodiments, the embedder comprises 10 layers to 20 layers, 10 layers to 30 layers, 10 layers to 40 layers, 10 layers to 50 layers, 10 layers to 60 layers, 10 layers to 70 layers, 10 layers to 80 layers, 10 layers to 90 layers, 10 layers to 100 layers, 10 layers to 200 layers, 20 layers to 30 layers, 20 layers to 40 layers, 20 layers to 50 layers, 20 layers to 60 layers, 20 layers to 70 layers, 20 layers to 80 layers, 20 layers to 90 layers, 20 layers to 100 layers, 20 layers to 200 layers, 30 layers to 40 layers, 30 layers to 50 layers, 30 layers to 60 layers, 30 layers to 70 layers, 30 layers to 80 layers, 30 layers to 90 layers, 30 layers to 100 layers, 30 layers to 200 layers, 40 layers to 50 layers, 40 layers to 60 layers, 40 layers to 70 layers, 40 layers to 80 layers, 40 layers to 90 layers, 40 layers to 100 layers, 40 layers to 200 layers, 50 layers to 60 layers, 50 layers to 70 layers, 50 layers to 80 layers, 50 layers to 90 layers, 50 layers to 100 layers, 50 layers to 200 layers, 60 layers to 70 layers, 60 layers to 80 layers, 60 layers to 90 layers, 60 layers to 100 layers, 60 layers to 200 layers, 70 layers to 80 layers, 70 layers to 90 layers, 70 layers to 100 layers, 70 layers to 200 layers, 80 layers to 90 layers, 80 layers to 100 layers, 80 layers to 200 layers, 90 layers to 100 layers, 90 layers to 200 layers, or 100 layers to 200 layers. In some embodiments, the embedder comprises 10 layers, 20 layers, 30 layers, 40 layers, 50 layers, 60 layers, 70 layers, 80 layers, 90 layers, 100 layers, or 200 layers. In some embodiments, the embedder comprises at least 10 layers, 20 layers, 30 layers, 40 layers, 50 layers, 60 layers, 70 layers, 80 layers, 90 layers, or 100 layers. In some embodiments, the embedder comprises at most 20 layers, 30 layers, 40 layers, 50 layers, 60 layers, 70 layers, 80 layers, 90 layers, 100 layers, or 200 layers.

In some embodiments, transfer learning is not used to generate the final trained model. For example, in cases when sufficient data is available, a model generated at least in part using transfer learning does not provide a significant improvement in predictions compared to a model that does not utilize transfer learning (e.g., when tested against a test dataset). Accordingly, in some embodiments, a non-transfer learning approach is utilized to generate a trained model.

### Computing Systems and Software

In some embodiments, a system as described herein is configured to provide a software application such as a polypeptide prediction engine (e.g., providing an encoder-decoder framework). In some embodiments, the polypeptide prediction engine comprises one or more models for predicting an amino acid sequence mapped to at least one function or property based on input data such as an initial seed amino acid sequence. In some embodiments, a system as described herein comprises a computing device such as a digital processing device. In some embodiments, a system as described herein comprises a network element for communicating with a server. In some embodiments, a system as described herein comprises a server. In some embodiments, the system is configured to upload to and/or download data from the server. In some embodiments, the server is configured to store input data, output, and/or other information. In some embodiments, the server is configured to backup data from the system or apparatus.

In some embodiments, the system comprises one or more digital processing devices. In some embodiments, the system comprises a plurality of processing units configured to generate the trained model(s). In some embodiments, the system comprises a plurality of graphic processing units (GPUs), which are amenable to machine learning applications. For example, GPUs are generally characterized by an increased number of smaller logical cores composed of arithmetic logic units (ALUs), control units, and memory caches when compared to central processing units (CPUs). Accordingly, GPUs are configured to process a greater number of simpler and identical computations in parallel, which are amenable to the math matrix calculations common in machine learning approaches. In some embodiments, the system comprises one or more tensor processing units (TPUs), which are AI application-specific integrated circuits (ASIC) developed by Google for neural network machine learning. In some embodiments, the methods described herein are implemented on systems comprising a plurality of GPUs and/or TPUs. In some embodiments, the systems comprise at least 2, 3, 4, 5, 6, 7, 8, 9, 10, 15, 20, 30, 40, 50, 60, 70, 80, 90, or 100 or more GPUs or TPUs. In some embodiments, the GPUs or TPUs are configured to provide parallel processing.

In some embodiments, the system or apparatus is configured to encrypt data. In some embodiments, data on the server is encrypted. In some embodiments, the system or apparatus comprises a data storage unit or memory for storing data. In some embodiments, data encryption is carried out using Advanced Encryption Standard (AES). In some embodiments, data encryption is carried out using 128-bit, 192-bit, or 256-bit AES encryption. In some embodiments, data encryption comprises full-disk encryption of the data storage unit. In some embodiments, data encryption comprises virtual disk encryption. In some embodiments, data encryption comprises file encryption. In some embodiments, data that is transmitted or otherwise communicated between the system or apparatus and other devices or servers is encrypted during transit. In some embodiments, wireless communications between the system or apparatus and other devices or servers is encrypted. In some embodiments, data in transit is encrypted using a Secure Sockets Layer (SSL).

An apparatus as described herein comprises a digital processing device that includes one or more hardware central processing units (CPUs) or general purpose graphics processing units (GPGPUs) that carry out the device's functions. The digital processing device further comprises an operating system configured to perform executable instructions. The digital processing device is optionally connected to a computer network. The digital processing device is optionally connected to the Internet such that it accesses the World Wide Web. The digital processing device is optionally connected to a cloud computing infrastructure. Suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein.

Typically, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

A digital processing device as described herein either includes or is operatively coupled to a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a system or method as described herein generates a database as containing or comprising input and/or output data. Some embodiments of the systems described herein are computer based systems. These embodiments include a CPU including a processor and memory which may be in the form of a non-transitory computer readable storage medium. These system embodiments further include software that is typically stored in memory (such as in the form of a non-transitory computer readable storage medium) where the software is configured to cause the processor to carry out a function. Software embodiments incorporated into the systems described herein contain one or more modules.

In various embodiments, an apparatus comprises a computing device or component such as a digital processing device. In some of the embodiments described herein, a digital processing device includes a display to display visual information. Non-limiting examples of displays suitable for use with the systems and methods described herein include a liquid crystal display (LCD), a thin film transistor liquid crystal display (TFT-LCD), an organic light emitting diode (OLED) display, an OLED display, an active-matrix OLED (AMOLED) display, or a plasma display.

A digital processing device, in some of the embodiments described herein includes an input device to receive information. Non-limiting examples of input devices suitable for use with the systems and methods described herein include a keyboard, a mouse, trackball, track pad, or stylus. In some embodiments, the input device is a touch screen or a multi-touch screen.

The systems and methods described herein typically include one or more non-transitory computer readable storage media encoded with a program including instructions executable by the operating system of an optionally networked digital processing device. In some embodiments of the systems and methods described herein, the non-transitory storage medium is a component of a digital processing device that is a component of a system or is utilized in a method. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Typically the systems and methods described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Typically, the systems and methods described herein include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

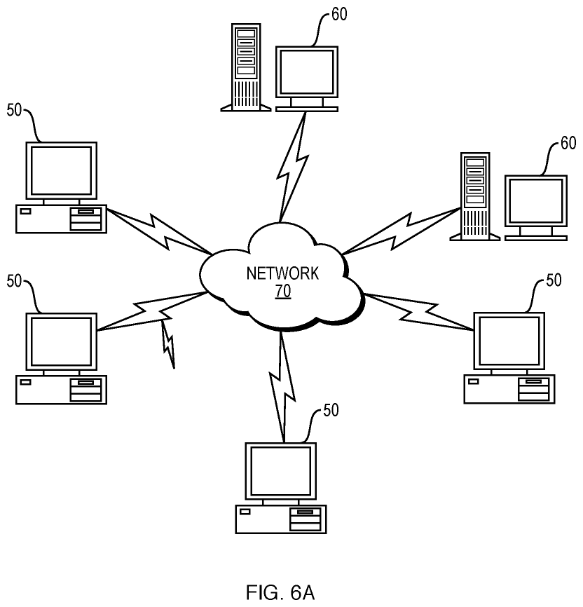

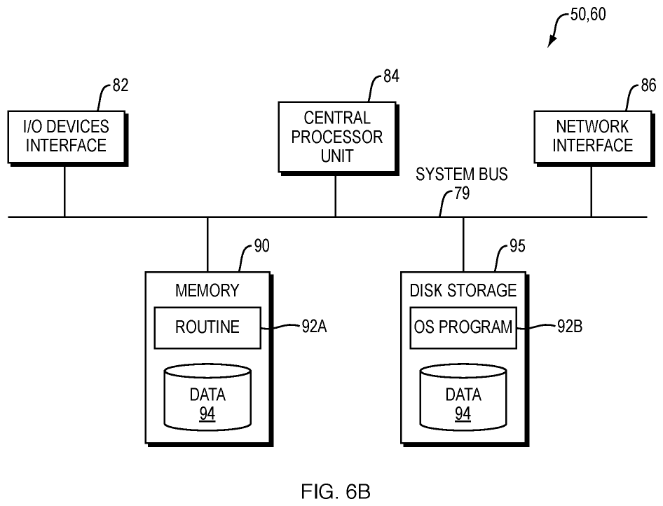

FIG. 6A illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

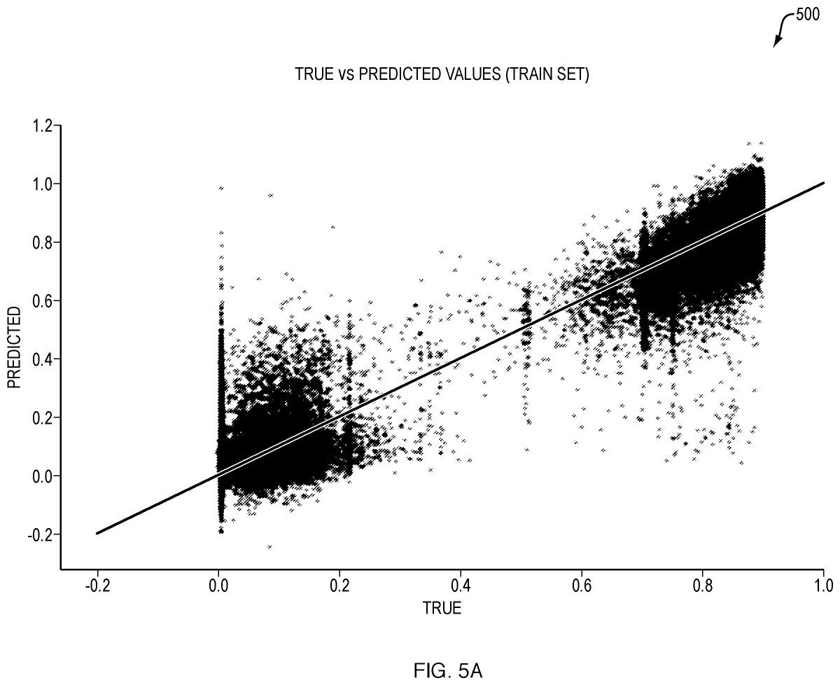

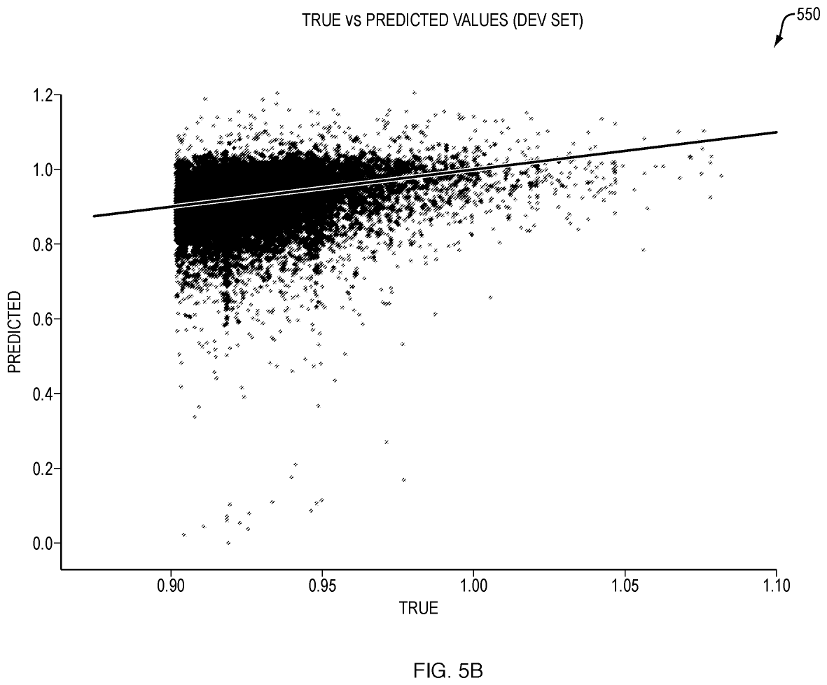

FIG. 6B is a diagram of an example internal structure of a computer (e.g., client processor/device 50 or server computers 60) in the computer system of FIG. 6A. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement an embodiment of the present invention (e.g., neural networks, encoder, and decoder detailed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

### Certain Definitions

As used herein, the singular forms “a”, “an” and “the” include plural references unless the context clearly dictates otherwise. For example, the term “a sample” includes a plurality of samples, including mixtures thereof. Any reference to “or” herein is intended to encompass “and/or” unless otherwise stated.

The term “nucleic acid” as used herein generally refers to one or more nucleobases, nucleosides, or nucleotides. For example, a nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO3) groups. A nucleotide can include a nucleobase, a five-carbon sugar (either ribose or deoxyribose), and one or more phosphate groups. Ribonucleotides include nucleotides in which the sugar is ribose. Deoxyribonucleotides include nucleotides in which the sugar is deoxyribose. A nucleotide can be a nucleoside monophosphate, nucleoside diphosphate, nucleoside triphosphate or a nucleoside polyphosphate. Adenine, cytosine, guanine, thymine, and uracil are known as canonical or primary nucleobases. Nucleotides having non-primary or non-canonical nucleobases include bases that have been modified such as modified purines and modified pyrimidines. Modified purine nucleobases include hypoxanthine, xanthine, and 7-methylguanine, which are part of the nucleosides inosine, xanthosine, and 7-methylguanosine, respectively. Modified pyrimidine nucleobases include 5,6-dihydrouracil and 5-methylcytosine, which are part of the nucleosides dihydrouridine and 5-methylcytidine, respectively. Other non-canonical nucleosides include pseudouridine (Ψ), which is commonly found in tRNA.

As used herein, the terms “polypeptide”, “protein” and “peptide” are used interchangeably and refer to a polymer of amino acid residues linked via peptide bonds and which may be composed of two or more polypeptide chains. The terms “polypeptide”, “protein” and “peptide” refer to a polymer of at least two amino acid monomers joined together through amide bonds. An amino acid may be the L-optical isomer or the D-optical isomer. More specifically, the terms “polypeptide”, “protein” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides. Amino acids include the canonical amino acids arginine, histidine, lysine, aspartic acid, glutamic acid, serine, threonine, asparagine, glutamine, cysteine, glycine, proline, alanine, valine, isoleucine, leucine, methionine, phenylalanine, tyrosine, and tryptophan. Amino acids can also include non-canonical amino acids such as selenocysteine and pyrrolysine. Polypeptides can be modified, for example, by the addition of carbohydrate, lipid, phosphorylation, etc., e.g., by post-translational modification, as well as combinations of the foregoing. Proteins can comprise one or more polypeptides. Amino acids include the canonical L-amino acids arginine, histidine, lysine, aspartic acid, glutamic acid, serine, threonine, asparagine, glutamine, cysteine, glycine, proline, alanine, valine, isoleucine, leucine, methionine, phenylalanine, tyrosine, and tryptophan. Amino acids can also include non-canonical amino acids such as the D-isomers of the canonical amino acids, as well as additional non-canonical amino acids, such as selenocysteine and pyrrolysine. Amino acids also include the non-canonical 0-alanine, 4-aminobutyric acid, 6-aminocaproic acid, sarcosine, statine, citrulline, homocitruline, homoserine, norleucine, norvaline, and ornithine. Polypeptides can also include post-translational modifications, including one or more of: acetylation, amidation, formylation, glycosylation, hydroxylation, methylation, myristoylation, phosphorylation, deamidation, prenylation (e.g., farnesylation, geranylation, etc.), ubiquitination, ribosylation and sulfation, including combinations of the foregoing. Accordingly, in some embodiments, a polypeptide provided by the invention or used in the methods or systems provided by the invention can, in different embodiments, contain: only canonical amino acids, only non-canonical amino acids, or a combination of canonical and non-canonical amino acids, such as one or more D-amino acid residues in an otherwise L-amino acid containing polypeptides.

As used herein, the term “neural net” refers to an artificial neural network. An artificial neural network has the general structure of an interconnected group of nodes. The nodes are often organized into a plurality of layers in which each layer comprises one or more nodes. Signals can propagate through the neural network from one layer to the next. In some embodiments, the neural network comprises an embedder. The embedder can include one or more layers such as embedding layers. In some embodiments, the neural network comprises a predictor. The predictor can include one or more output layers that generate the output or result (e.g., a predicted function or property based on a primary amino acid sequence).

As used herein, the term “artificial intelligence” generally refers to machines or computers that can perform tasks in a manner that is “intelligent” or non-repetitive or rote or pre-programmed.

As used herein, the term “machine learning” refers to a type of learning in which the machine (e.g., computer program) can learn on its own without being programmed.

As used herein, the phrase “at least one of a, b, c, and d” refers to a, b, c, or d, and any and all combinations comprising two or more than two of a, b, c, and d.

### Examples

### Example 1: In Silico Engineering A Green Fluorescent Protein Using Gradient-Based Design

An in silico machine learning approach was used to transform a protein that did not glow into a fluorescent protein. The source data for this experiment was 50,000 publicly available GFP sequences for which fluorescence had been assayed. First, an encoder neural network was generated with the assistance of transfer learning by using a model that was first pre-trained on the UniProt database and then taking the model and training it to predict fluorescence from the sequence. The proteins in the lower 80% of brightness were selected as the training data set, while the top 20% brightest proteins were withheld as a validation data set. The mean squared error on the training and validation sets were <0.001, indicating high accuracy to predict fluorescence directly from sequence. Data plots showing the true vs. predicted fluorescence values in the training and validation sets is shown in FIG. 5A and FIG. 5B, respectively.

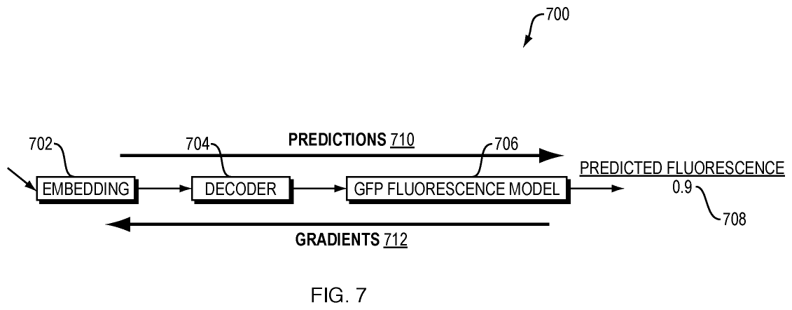

FIG. 7 shows a diagram illustrating the gradient-based design (GBD) for engineering a GFP sequence. The embedding 702 is optimized based on the gradients. The decoder 704 is used to determine the GFP sequence based on the embedding, after which the GFP sequence can be assessed by the GFP fluorescence model 706 to arrive at the predicted fluorescence 708. As shown in FIG. 7, the process of generating the GFP sequence using gradient-based design includes: taking one step in embedding space as guided by the gradients, making a prediction 710, re-evaluating the gradient 712, and then repeating this process.

After the encoder was trained, a sequence that did not currently fluoresce was selected as the seed protein and projected into embedding space (e.g., a 2-dimensional space) using the trained encoder. A gradient based update procedure was run to improve the embedding, thus optimizing the embedding from the seed protein. Next, derivatives were calculated and used to move through embedding space towards a region of higher function The optimized embedding coordinates were improved with respect to the fluorescence function. Once the desired level of function was achieved, the coordinates in embedding space were projected back into protein space, resulting in a sequence of amino acids with the desired function.

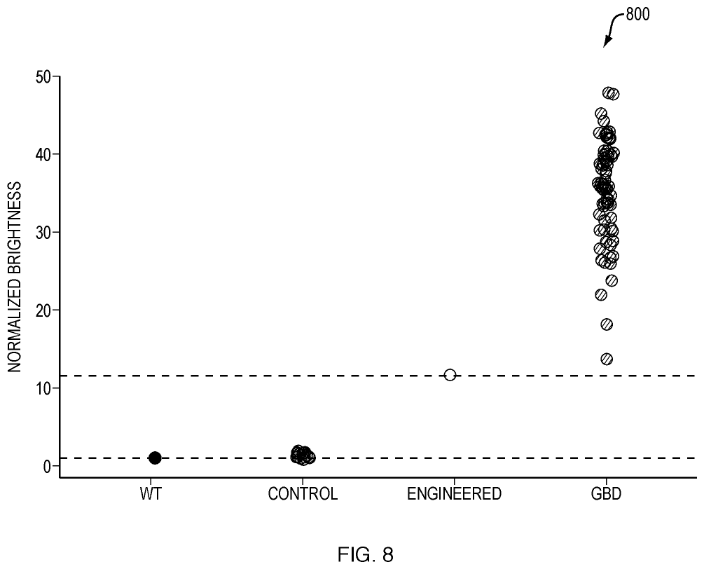

A selection of 60 of the GBD-designed sequences with the highest predicted brightness was selected for experimental validation. Results for the experimental validation of the sequences created using GBD are shown in FIG. 8. The Y-axis is fold-change in fluorescence relative to avGFP (WT). FIG. 8 shows, from left to right: (1) WT—brightness of avGFP, which is a control for all of the GFP sequences that the supervised model was trained on; (2) Engineered: A human designed GFP known as ‘super folder’ (sfGFP); (3) GBD: novel sequences created using the gradient-based design procedure. As can been seen, in some instances the sequences designed by GBD are ˜50 times brighter than the wild-type and training sequences, and 5 times brighter than the well-known human engineered sfGFP. These results validate GBD as being capable of engineering polypeptides having a function that is superior to that of human engineered polypeptides.

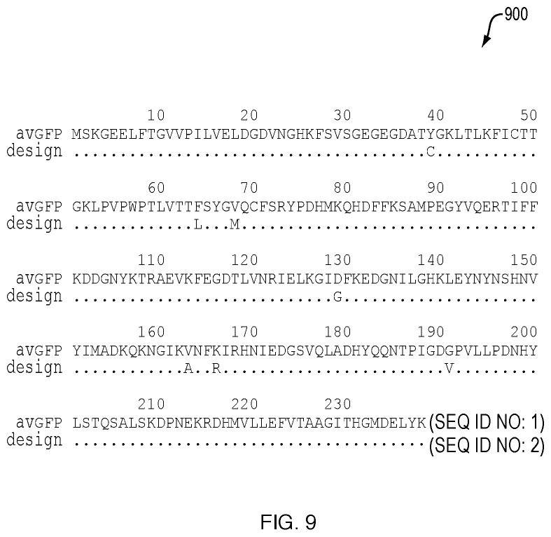

FIG. 9 shows a pairwise amino acid sequence alignment 900 of avGFP against the GBD-engineered GFP sequence with the highest experimentally validated fluorescence, which was approximately 50 times higher than avGFP. A period ‘.’ indicates no mutation relative to avGFP, while mutations or pairwise differences are shown by the single letter amino acid code representing the GBD-engineered GFP amino acid residue at the indicated location in the alignment. As shown in FIG. 9, the pairwise alignment reveals 7 amino acid mutations or residue differences between avGFP, which is SEQ. NO. 1, and the GBD-engineered GFP polypeptide sequence, which can be referred to as SEQ. NO. 2.

The avGFP is a 238 amino acid long polypeptide having the following sequence of SEQ ID NO:1 The GBD-engineered GFP polypeptide has 7 amino acid mutations relative to the avGFP sequence: Y39C, F64L, V68M, D129G, V163A, K166R, and G191V.

The residue-wise accuracy of the decoder was >99.9% on both the training and validation data, which meant that, on average, the decoder made 0.5 mistakes per GPF sequence (given that GFP is 238 amino acids long). Next, the decoder was evaluated for its performance with respect to protein design. First, each protein in the training and validation sets was embedded using the encoder. Next, those embeddings were decoded using the decoder. Finally, the fluorescence values of the decoded sequence were predicted using the encoder, and these predicted values were compared to the values predicted using the original sequence. A summary of this process is shown in FIG. 4.

The correlation between the predicted values from the original sequence and the predicted values from the decoded sequences was computed. High levels of agreement were observed in both the training and validation data sets. These observations are summarized in Table 1.

TABLE 1

| Data | Correlation |
|---|---|
| Training | 0.99 |
| Validation | 0.77 |

### Example 2 in Silico Engineering a Beta Lactamase Gene Using Gradient-Based Design

An in silico machine learning approach was used to transform a beta-lactamase to gain resistance to an antibiotic that it was not previously resistant to. Using a training set of 662 publicly available beta-lactamase sequences for which resistance to 11 antibiotics had been measured, a multi-task deep learning model was built to predict resistance to these antibiotics on the basis of amino acid sequence.

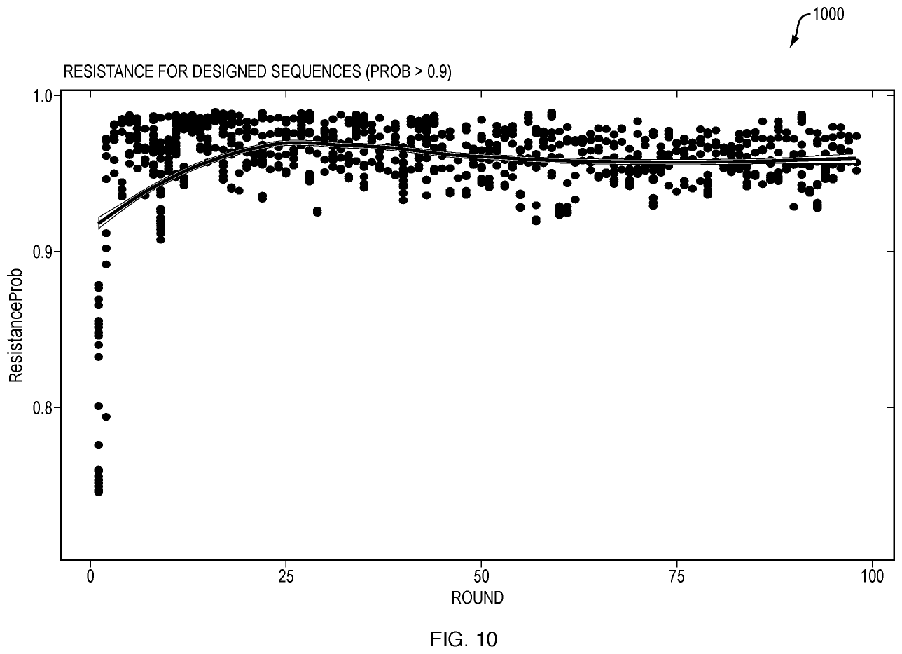

Next, 20 beta-lactamases were selected from the training set that were not resistant to a test antibiotic with the goal of designing new sequences that would be resistant to this antibiotic. Gradient-based design (GBD) was applied to these sequences for a total 100 iterations. A visualization of this process is shown in FIG. 10. As detailed previously, an initial sequence was used as a seed that was mapped onto the embedding space and subsequently optimized through the 100 iterations. FIG. 10 shows the predicted resistance to test antibiotic for designed sequences as a function of gradient-based design iteration. The y-axis indicates the resistance predicted by the model, and the x-axis indicates the rounds or iterations of gradient-based design as the embedding was optimized. FIG. 10 illustrates how the predicted resistance increased through the rounds or iterations of GBD. The seed sequences started with low resistance (round 0) and were iteratively improved to have high predicted resistance (probability >0.9) after several rounds. As shown, it appears the predicted resistance peaked by about 25 rounds and then plateaued.

Unlike GFP, beta-lactamases have variable length, and therefore, the length of the protein is something GBD is able to control in this example.

A selection of 7 sequences was made for experimental validation, which are shown in Table 2 below.

TABLE 2
Seven sequences designed by GBD were selected for experimental
validation. These seven sequences were selected for a combination of
having a high probability of resistance to test antibiotic
(ResistanceProb), having low sequence identity to sequences that were
resistant to test antibiotic in the training data (ClassPercentID), and for
having low mutual sequence identity. The longest beta-lactamase in
the training data was 400 amino acids, a length which was exceeded by
several of the GBD-designed beta lactamase polypeptide sequences.

| ResistanceProb | ClassPercentID | Length |
|---|---|---|
| 0.96605885 | 74.83870968 | 449 |
| 0.989722192 | 99.18478261 | 368 |
| 0.965560615 | 90.34653465 | 404 |
| 0.958946645 | 76.14457831 | 366 |
| 0.973307133 | 82.87841191 | 373 |
| 0.96702373 | 82.25 | 370 |
| 0.953287661 | 81.51447661 | 449 |

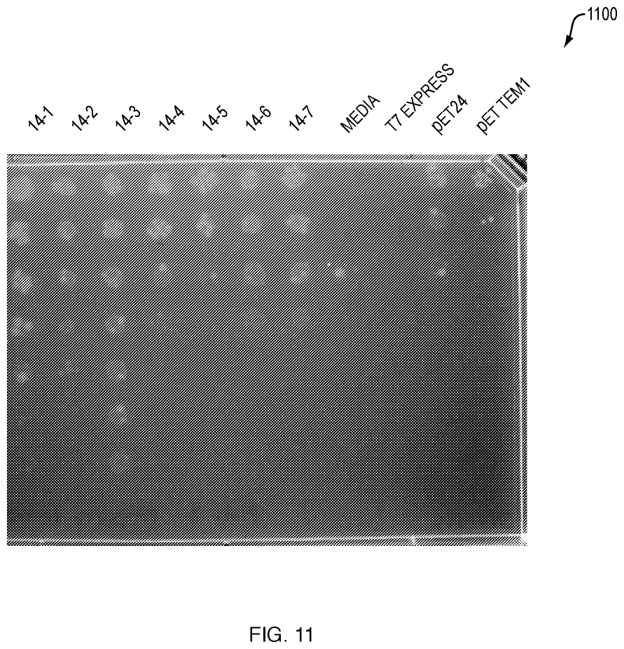

A validation experiment was performed for seven novel beta-lactamases designed using GBD. Bacteria transformed with vectors expressing the beta-lactamases underwent 10-fold serial dilution, and were grown in agar plates in the presence of 8 ug/ml test antibiotic+1 mM IPTG. FIG. 11 is a diagram illustrating a test of antibiotic resistance. The canonical beta-lactamase, TEM-1, is shown in the last column. As is evident, several of the designed sequences show great resistance ability to the test antibiotic than TEM-1. The beta-lactamases at columns 14-1 and 14-2 have colonies five spots down. Column 14-3 has colonies seven spots down. Column 14-4, 14-6, and 14-7 have colonies four spots down. Column 14-5 has colonies three spots down. Meanwhile, TEM-1 only has colonies two spots down.

### Example 3—Synthetic Experiments Using Gradient Based Design on Simulated Landscapes

Computational design of biological sequences with specific functional properties using machine learning is a goal of this disclosure. A common strategy is model-based optimization: a model that maps sequence to function is trained on labeled data and subsequently optimized to produce sequences with the desired function. However, naive optimization methods fail to avoid out-of-distribution inputs on which the model error is high. To address these issues, explicit and implicit methods constrain the objective to in-distribution inputs, which efficiently generates novel biological sequences.

Protein engineering refers to the generation of novel proteins with desired functional properties. The field has numerous applications including design of protein therapeutics, agricultural proteins and industrial biocatalysts. Identifying amino-acid sequences that code for proteins with specified function is challenging partly because the space of candidate sequences is combinatorially large, while the subset of functional sequences is vanishingly small.

One family of methods that has seen success is directed evolution: an iterative process which alternates between sampling from a library of genetic variants and screening for those with improved function from which to build the next round of candidates. Even with the development of high-throughput assays, the process is time and resource intensive, requiring many iterations and screening of large numbers of variants. In many applications, designing high-throughput assays for a desired functional property is challenging or infeasible.

Recent approaches leverage machine learning methods to design libraries more efficiently and arrive at higher fitness sequences with fewer iterations/screens. One such method is model-based optimization. In this setting, a model mapping sequence to function is fit to labeled data. The model then computationally screens variants and design higher fitness libraries. In an embodiment, the system and method of the disclosure ameliorates problems that arise in naïve approaches to model-based optimization and improves generated sequence.

In an example, let X denote the space of protein sequences and ƒ be a real-valued map on protein space encoding a property of interest (e.g, fluorescence, activity, expression, solubility). The task of designing a novel protein with a specified function can then be reformulated as finding solutions to:

$${argmax}_{x ∈ X} ( f ( x ) ) \qquad ( 1 )$$

where ƒ is in general unknown. This class of problems is referred to as model-based optimization. This problem can be restricted to a static setting, in which one cannot query f directly but is provided a labeled dataset D=(xᵢ,yᵢ)ᵢ₌₁^{N} where the labels y are possibly noisy: yᵢ≈ƒ(xᵢ).

A naive approach is to use D to fit a model ƒ_{θ} approximating ƒ and then solve:

$${argmax}_{x ∈ X} ( {f}_{θ} ( x ) ) \qquad ( 2 )$$

This tends to produce poor results as an optimizer can find points in such that ƒ_{θ} is erroneously large. A key problem is that the space of possible amino acid sequences has very high dimension, but the data is typically sampled from a much lower dimensional subspace. This is exacerbated by the fact that in practice θ is high-dimensional and ƒ_{θ} highly non-linear (e.g. due to phenomenon like epistasis in biology). Therefore, the output must be constrained in some way to restrict the search to a class of admissible sequences on which ƒ_{θ} is a good approximation of ƒ.

One approach is to fit a probabilistic model p_{θ} to (xᵢ)^{N} such that p_{θ} (x) is the probability that a sequence x is sampled from the data distribution. Some examples of model classes for which likelihoods can be explicitly computed (or lower-bounded) are first-order/sitewise models, hidden Markov models, conditional random fields, variational auto-encoders (VAEs), auto-regressive models, and flow-based models. In an embodiment, the method optimizes the function:

$${argmax}_{x ∈ X} ( {f}_{θ} ( x ) + λ {p}_{θ} ( x ) ) \qquad ( 3 )$$

where λ>0 is a fixed hyperparameter. Often labeled data are expensive or scarce, but unlabeled examples of proteins from a family of interest are readily available. In practice, p_{θ} can be fit to a larger dataset of unlabeled proteins from this family.

One challenge to optimizing directly in sequence space is that sequence space is discrete, making it unsuitable for gradient-based methods. Leveraging the fact that ƒ_{θ} is a smooth function of a learned continuous representation of sequence-space can make use of gradients and optimize more efficiently. To that end, ƒ_{θ}=a_{θ} e_{θ} where ƒ_{θ} is an L layer neural network, e_{θ}: Z, referred to as the encoder, is the first K layers, and a_{θ}:Z→R, referred to as the annotator is the last L−K layers. This enables us to move optimization to the space Z and make use of gradients. The unregularized analog is to solve:

$$z * := {argmax}_{z ∈ Z} {a}_{θ} ( z ) \qquad ( 4 )$$

Then fit a probabilistic decoder d_{φ}:Z→p(X) mapping z→d_{φ}(x|z) such that

$${d}_{φ}^{*} ( {x}^{′} ) := {argmax}_{x} {d}_{φ} ( x \vert {e}_{θ} ( {x}^{′} ) ) \approx {x}^{′}$$

for x′ sampled from the data distribution, which can return d*_{ϕ}(z*). One may expect that problems here will compound, as gradients may pull z* into areas of Z where not only a_{θ} but also d_{φ} have high error. The method is motivated by the observation that since a_{θ} and d_{φ} are trained on the same data manifold, reconstruction error of d_{φ} tends to correlate with mean absolute error of a_{θ}. An objective function as follows is proposed:

$${argmax}_{z ∈ Z} {f}_{θ} ( {d}_{ϕ} ( x \vert z ) ) \qquad ( 5 )$$

This adds an implicit constraint to the optimization. Stable solutions to (5) correspond to areas of Z where d_{φ} (x z) has low entropy and low reconstruction error. A heuristic for thinking about this regularization is that, because the decoder is trained to output distributions on that are concentrated on points in the data distribution, the mapping z→e_{θ} (d_{φ}(x|z)) can be considered a projection onto the data manifold. While the earlier ƒ_{θ} was a map on X, and equation suggests ƒ_{θ} is be a map on p( ). Below, a natural extension of ƒ_{θ} to p( ) for which equation (5) fits is described, however. Finally, as with p_{θ} in equation (3), the decoder d_{φ} can be fit to a larger unlabeled dataset of proteins from the family of interest if available using gradient ascent as Gradient Based Design (GBD) via equation (5).

Results—Synthetic Experiments

Evaluating model-based optimization methods requires querying the ground truth function ƒ. In practice, this can be slow and/or expensive. To aid with the development and evaluation of methods, the method is tested with synthetic experiments in two settings: a lattice-protein optimization task and an RNA optimization task. In both tasks, the ground truth ƒ is highly nonlinear and approximate non-trivial biophysical properties of real biological sequences.

Lattice protein refers to the simplifying assumption that an L-length protein is restricted to take on conformations that lie on a 2-dimensional lattice with no self-intersections. Under this assumption one can enumerate all possible conformations and compute the partition function exactly, making many thermodynamic properties efficiently computable. A ground-truth fitness ƒ is defined as the free energy of an amino acid chain with respect to a fixed conformation s_{ƒ} Optimizing sequences with respect to this fitness amounts to finding sequences that are stable with respect to a fixed structural conformation, a longstanding goal in sequence design.

The free energy of a nucleotide sequence with respect to a fixed conformation can be computed efficiently without many of the simplifying assumptions made in 2-D lattice protein models. In the RNA optimization setting, ƒ is defined on the space of nucleotide sequences as the free energy with respect to a fixed conformation s_{ƒ} of a known tRNA structure.

For both tasks, after ƒ is defined, a fitness landscape from which to select training data is generated by modified Metropolis-Hastings sampling. Under Metropolis-Hastings, the probability of a sequence x being included in the landscape is asymptotically proportional to ƒ (x). The data is split according to fitness: validation data are sampled uniformly from higher fitness sequences and training data from lower fitness sequences to evaluate methods on their ability to generate sequences with fitness greater than seen during training, a desirable property in real-world applications.

A convolutional neural network ƒ_{θ} and a site-wise p_{θ} are fit to the data. A cohort of 192 seed sequences are sampled from the training data and optimized according to discrete optimization objectives (2) and (3) and gradient-based optimization objectives (4) and (5). Discrete objectives are optimized by a greedy local search algorithm in which at each step a number of candidate mutations are sampled from an empirical distribution given by the training data, and the best mutation according to the objective is selected for each sequence in the cohort.

Naive optimization quickly drives the cohort to areas of space where model error is high and fails to improve the average fitness of the cohort in both experiments. Regularization can reduce this effect, allowing the average fitness of the cohort to improve while model error is kept low. Few sequences generated (<1%) exceed fitness values seen during training on either task.

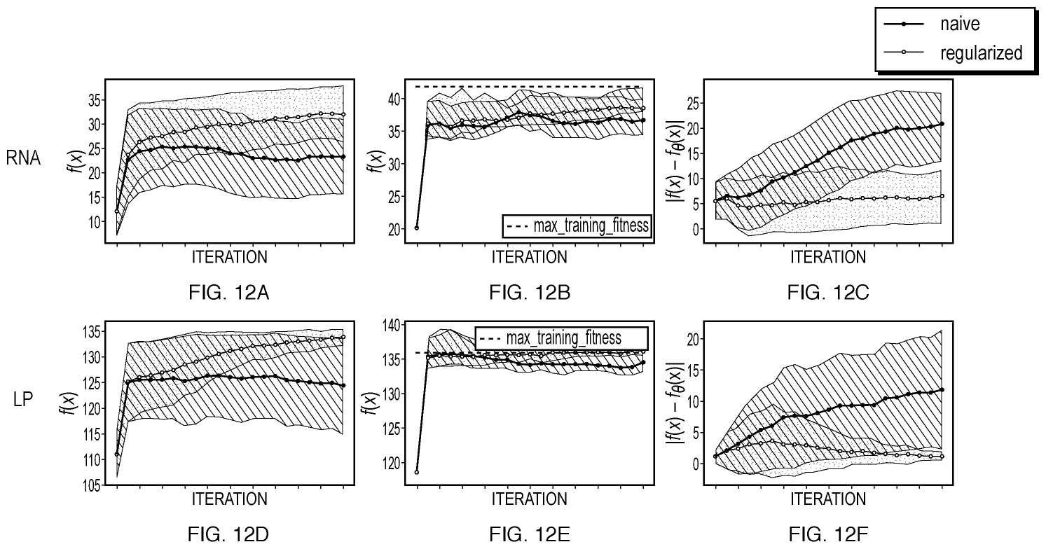

FIG. 12A-F are graphs illustrating discrete optimization results on RNA optimization (12A-C) and lattice-protein optimization (12D-F). FIGS. 12A and 12D illustrate fitness (μ±σ) across the cohort during optimization. Naive optimization does not result in a meaningful increase in mean fitness in either environment, while regularized objective is able to do so. FIGS. 12B and 12E illustrate the fitness of the sub-cohort consisting of top 10 percentile in fitness (shaded min to max performance in sub-cohort). Sequences with meaningfully higher fitness than seen during training cannot be found by either method in the RNA sandbox. FIGS. 12C and 12F illustrate the absolute deviation (μ+σ) of ƒ_{θ} from ƒ across the cohort during optimization. The naive objective fails to improve cohort performance because the cohort moves into parts of space where the model is unreliable.

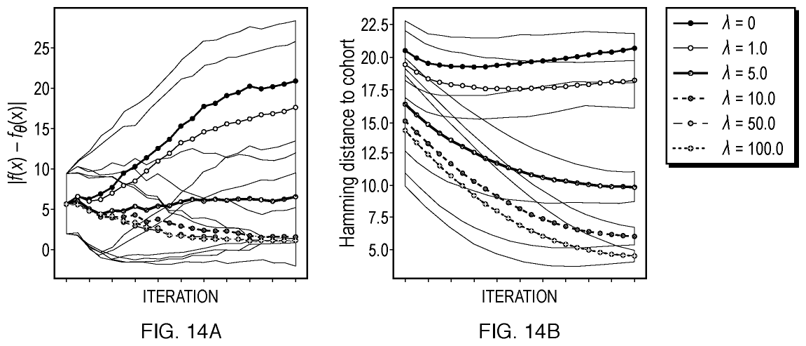

FIG. 14 illustrates the effect of up-weighting the regularization term λ in equation (3): larger λ results in decreased model error but a corresponding decrease in sequence diversity over the course of optimization as the model is restricted to sequences that are assigned high probability by p_{θ}. For all experiments testing this system, λ is set to 5 if not otherwise specified. However, other values could be used for other tests. The left graph illustrates mean model error (μ+σ) across cohort decreases as λ is increased in objective (3), while the right graph illustrates sequence diversity in the cohort decreases as well. Data taken from lattice-proteins sandbox environment. Gradient-based methods quickly move much further into space than discrete methods. GBD is able to explore regions of sequence space much further from initial seeds while maintaining comparably low model error to discrete regularized methods.

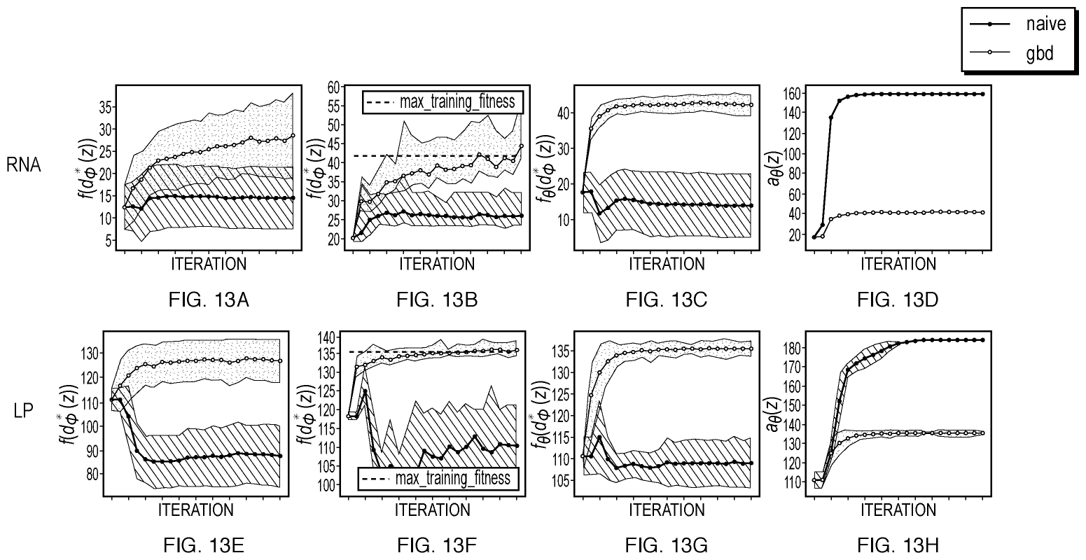

FIGS. 13A-H illustrate results for gradient-based optimization. The problems highlighted above when optimizing in are only exacerbated when working in Z: without regularization not only is the cohort driven to points z where a_{θ}(z) have unrealistically (and incorrectly) high predicted fitness values, but also the decoded sequences d*_{φ}(z) are not predicted to have high fitness by ƒ_{θ}. In both settings, naive optimization fails to improve mean fitness across the cohort and fails to find sequences that exceed fitness seen during training. GBD does not exhibit this behavior: successfully optimizing ƒ_{θ} d*, a_{θ}, and ƒ d*_{φ}. In both settings, GBD improves mean fitness of the cohort and the top 10% of sequences in the cohort consistently have fitness exceeding those seen during training.

FIGS. 13A-D illustrate gradient-based optimization results on RNA optimization and FIGS. 13E-H illustrate lattice-protein optimization. FIGS. 13A and 13E illustrate ƒ(d*_{φ}(z)) (μ±σ), the true fitness of the maximal-likelihood decoded sequence across the cohort during optimization. Naive optimization does not result in a meaningful increase in mean fitness in RNA sandbox and incurs a significant decrease in cohort fitness in the lattice-proteins environment. GBD is able to successfully improve mean cohort fitness during optimization. FIGS. 13B and 13F illustrate fitness of the sub-cohort consisting of top 10 percentile in fitness (shaded min to max performance in sub-cohort). GBD reliably finds sequences with fitness values exceeding those seen during training. FIGS. 13C and 13G are a panel illustrating ƒ_{θ}(d*_{φ}(z)) (μ±σ) of the cohort during optimization, the predicted fitness of the decoded sequence at the current point in Z. FIGS. 13D and 13H illustrate a_{θ}(z) (μ±σ) of the cohort during optimization, the predicted fitness of the current representation in Z. The naive objective quickly hyper-optimizes the a_{θ}, pushing the cohort to unrealistic parts of Z-space that cannot be decoded by d*_{φ} into meaningful sequences. The GBD objective successfully prevents this pathology.

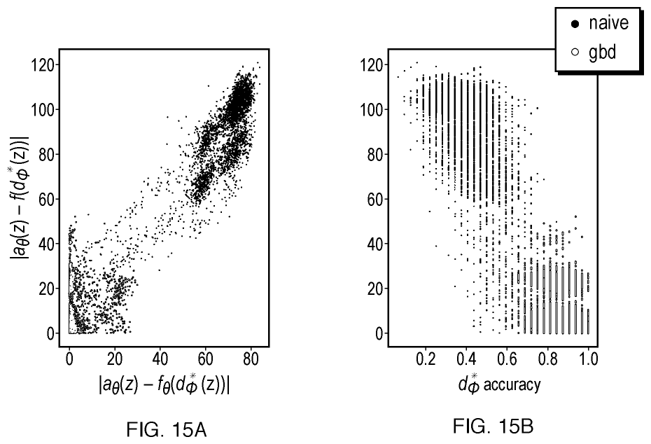

FIGS. 15A-B illustrates the heuristic motivating GBD: it drives the cohort to areas of Z where d*_{φ} can decode reliably. Viewed in X, this means d*_{ϕ}ºe_{θ} is approximately identity (right), or viewed in Z that ∥e_{Θ}ºd*_{ϕ}(z)−z∥ is small and hence ∥a_{θ}(z)−ƒ_{θ}ºd*_{ϕ}(z)−z∥ is small. The data suggests that ƒ_{θ} is also reliable in this area of space, as ƒ_{θ} and d_{φ} are trained on the same distribution.

FIG. 15A is a scatterplot of deviation of a_{θ}(z) from ƒ_{θ}(d*_{φ}(z)) plotted against deviation of a_{θ}(z) from ƒ(d*_{φ}(z)) over all steps of and all sequences in the cohort optimized in the lattice-proteins landscape. FIG. 15B is a graph illustrating the accuracy of dip, the maximal likelihood decoding of a point in Z plotted against deviation of a_{θ}(z) from ƒ(d*_{φ}(z)) on the same data. GBD provides regularization implicitly by pushing the cohort to areas of Z where d_{φ} decodes reliably. Since ƒ_{θ} and d_{φ} are fit on the same distribution, predicted fitness in this region is reliable.

In synthetic experiments, GBD is able to meet or exceed the performance of the monte carlo optimization methods explored in terms of fitness (mean and max) of the cohort. In practice GBD is much faster: discrete methods involve generating and evaluating K candidate mutations at every iteration. This requires K forward passes of the model per sequence per iteration. GBD requires one forward and one backward pass per sequence per iteration.

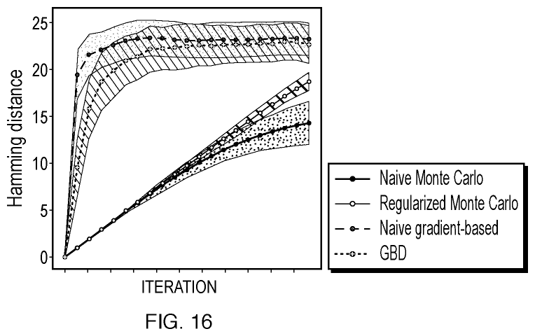

Additionally, FIG. 16 illustrates the number of mutations (μ±σ) from initial seed in cohort during optimization of various objectives in the lattice-proteins. FIG. 16 illustrates that GBD is able to find optima further away from initial seed sequences than discrete methods while maintaining a comparably low error.

Table 3 provides a comparison of all methods discussed as well as a random search baseline. On the RNA sandbox, GBD is the only method explored that could generate sequences with fitness greater than seen in the entire landscape generated by Metropolis Hastings (run for orders of magnitude more iterations than the optimization). The python package LatticeProteins enumerates all possible non self-intersecting conformations of a length-16 amino acid chain. This enumeration is used to compute free energies of length 16 amino acid chains under a fixed conformation sf A fitness function ƒ is defined on the space of length 32 amino-acid sequences as follows:

$$f ( x ) = E ( {x}_{1} ) + E ( {x}_{2} ) - R ( {x}_{1} , {x}_{2} ) \qquad ( 6 )$$

where E(x₁) is the free energy of the chain formed by the first 16 amino acid residues with respect to s_{ƒ}, E(x₂) is the free energy of the chain formed by the latter 16 amino acids residues with respect to s_{ƒ}

$$R ( {x}_{1} , {x}_{2} ) = c ( {( {x}_{1} )}_{i} , {( {x}_{2} )}_{i} ) \qquad ( 7 )$$

and c(α, β) are constant interaction terms sampled from a standard normal for all amino acids α, β.

RNA Structure Fitness Function

Let s_{ƒ} be a fixed tRNA structure. With the aid of the python package ViennaRNA, the fitness function ƒ is defined on the space of length-70 nucleotide sequences as:

$$f ( x ) = E ( x ) - min ( exp ( β d ( {s}_{f} , {s}_{x} ) ) , 20 ) \qquad ( 8 )$$

where d denotes hamming distance, β=0.3 is a hyperparameter, sₓ denotes the minimum energy conformation of x, and E(x) denotes the free energy of the sequence in conformation sₓ.

Greedy Monte Carlo Search Optimization

The method optimizes objectives 2 and 3 by a greedy monte carlo search algorithm. With x being a length L sequence, at each iteration, K mutations are sampled from a prior distribution given by the training data. More precisely, K positions are sampled uniformly from 1 . . . L with replacement, and for each position an amino acid (or nucleotide in the case of RNA optimization) is sampled from the marginal distribution given by the data at that position. The objective is then evaluated at each variant in the library (with the original sequence included) and the best variant is selected. This process is continued for M steps.

D. Generation of Fitness Landscapes

Given access to a fitness function ƒ on it is desirable to obtain samples on which to train a supervised model ƒ_{θ}. Uniformly sampling is infeasible due to the high dimensionality of X, intuitively because with high probability a sequence selected at random will have vanishingly low fitness. The goal is to obtain samples from a distribution whose density is proportional to ƒ. For each inner loop in the process, a cohort of M sequences is initialized randomly. For each sequence, drawn N mutations are drawn uniformly at random and include all MN sequences in the landscape. With (xᵢⱼ)^{N} denoting the N variants of sequence i, the method updates by sampling a mutation from a categorical distribution on [1 . . . N] with logits given by (ƒ(xᵢⱼ))^{N}. The inner loop is run for J steps, and C outer loops are run, as described further below.

Gradient Based Design

Gradient based design refers to the optimization of objective (4) by gradient ascent. Given ƒ_{θ}, d_{φ} and initial point z₀, set h:=ƒ_{θ} d_{φ}, an iterations of GBD consist of K steps of a gradient-based optimizer such as Adam to maximize h, followed by a decoding step where z e_{θ}(d_{φ}(z)). In practice, there is an effective learning rate is critical for good performance, a value of 0.05 was used throughout experiments with a K of 20.

Model Architectures and Training

The method factorizes ƒ_{θ}=a_{θ} e_{θ}. A convolutional encoder e_{θ} was used throughout all experiments consisting of alternating stacks of convolutional blocks and average pooling layers. A block comprises two layers wrapped in a residual connection. Each layer comprises a 1d convolution, a layer normalization, dropout, and a ReLU activation. A 2-layer fully connected feedforward network a_{θ} is used throughout. The decoder network, d_{φ} comprises of stacks of alternating residual blocks and transposed convolutional layers followed by a 2-layer fully connected feedforward network.

Parameter estimation is done sequentially rather than jointly: first ƒ_{θ} is fit, then the parameters θ are frozen and d_{φ} is fit. Learning is done by stochastic gradient descent to minimize MSE and cross entropy for ƒ_{θ}, d_{φ} respectively with an ADAM optimizer. ƒ_{θ} is fit for 20 epochs and d_{φ} for 40 epochs using a one-cycle learning rate annealing schedule with a maximal learning rate of 10⁻⁴. After each epoch model parameters are saved and after training the best parameters as measured by validation loss are selected for generation. A site-wise p_{θ} is used in all experiments which is fit by maximum likelihood.

A variational auto-encoder was fit to data by maximizing the evidence lower bound. Encoder and decoder parameters are learned jointly by way of re-parameterization (amortization). A constant learning rate of 10⁻³ was used for 50 epochs with early-stopping set and a patience parameter of 10. For 20 iterations N=5000 sequences are sampled from the standard normal prior and passed through the decoder, assigned predicted fitness by ƒ_{θ}. The VAE is fine-tuned for 10 epochs on these sequences, re-weighted to generate sequences with higher predicted fitness. Results in table I are reported for the iteration corresponding to maximum mean true fitness for both methods as both generative models collapse to delta mass functions before 20 iterations is complete. Thus metrics reported encapsulate peak performance of the methods.

TABLE 3
Comparison of methods on lattice-proteins optimization and RNA
optimization: For methods random search, naive
monte carlo, regularized monte carlo, naive gradient-based and
gradient-based design: (μ ± σ) of true fitness of full cohort being optimized,
top 10% of the cohort, and maximal fitness sequence in cohort at the
end of optimization. Optimization consists of 20 iterations applied to 192
sequences sampled from training data (kept constant across method).

| | Lattice Proteins | | | RNA | | |
|---|---|---|---|---|---|---|
| Method | Full cohort | Top 10% | Max | Full cohort | Top 10% | Max |
| Random | 59.96 ± 11.42 | 81.62 ± 6.36 | 93.87 | 2.12 ± 4.73 | 11.67 ± 2.54 | 18.29 |
| Search | | | | | | |
| Naïve | 124.44 ± 9.70 | 134.47 ± 1.10 | 136.65 | 23.41 ± 7.64 | 36.77 ± 2.18 | 41.68 |
| monte | | | | | | |
| carlo | | | | | | |
| Regularized | 133.87 ± 1.62 | 136.09 ± 0.48 | 137.22 | 32.07 ± 5.71 | 38.55 ± 0.71 | 39.98 |
| Monte | | | | | | |
| Carlo | | | | | | |
| Gradient | 87.80 ± 12.55 | 110.76 ± 5.93 | 121.81 | 28.32 ± 9.53 | 44.43 ± 4.49 | 58.5 |
| Based | | | | | | |
| Design | | | | | | |

### Example 4 In Silico Engineering an Antibody Using Gradient-Based Design

The foregoing describes generation of an antibody that binds fluorescein isothiocyanate (FITC) with improved dissociation constant (KD), using gradient-based design. Models were trained on a publicly available dataset of KD estimates for a library of 2825 unique antibody sequences, measured using fluorescence-activated cell sorting followed by next generation sequencing as described in Adams R M; Mora T; Walczak A M; Kinney J B, Elife, “Measuring the sequence-affinity landscape of antibodies with massively parallel titration curves” (2016) (hereinafter “Adams et al.”), which is hereby incorporated by reference in its entirety. This dataset of sequence and KD pairs mapping antibody sequences to KD was split in three ways. The first split was made by holding out top 6% of performing sequences for validation (so model is trained on lowest 94%). The second split was made by holding out top 15% of performing sequences for validation (so model is trained on lowest 85%) Third split was made by sampling uniformly (iid) 20% of the sequences to be held out for validation.

For each split, a supervised model including an encoder (mapping sequence to embedding) and annotator (mapping embedding to KD) is fit jointly. A decoder is then fit on the same training set mapping embedding back to sequence. For each model, 128 seeds are sampled uniformly from the training set and optimized in two ways. The first way is that for 5 rounds by GBD, each round consisting of 20 GBD steps followed by a projection back through the decoder. The second way is for 5 rounds by GBD+, (where the objective is augmented with a first-order regularization) each round consisting of 20 GBD steps followed by a projection back through the decoder. GBD+ uses additional regularization, including constraining the method using an MSA (multiple sequence alignment). Thus each model yields two cohorts of candidates (one for each method, GBD, GBD+). Final sequences to order are selected from each cohort by first labeling each candidate with a predicted expression (from an independently trained expression model, fit to a dataset of (sequence, expression data split in an i.i.d (independent and identically distributed) manner). Cohorts are filtered in two ways: a sequence is removed if it is predicted to have low expression, and a sequence is removed if it's predicted fitness is lower than its seeds initial predicted fitness. Of the remaining sequences, highest predicted fitness sequences were chosen to measure in lab.

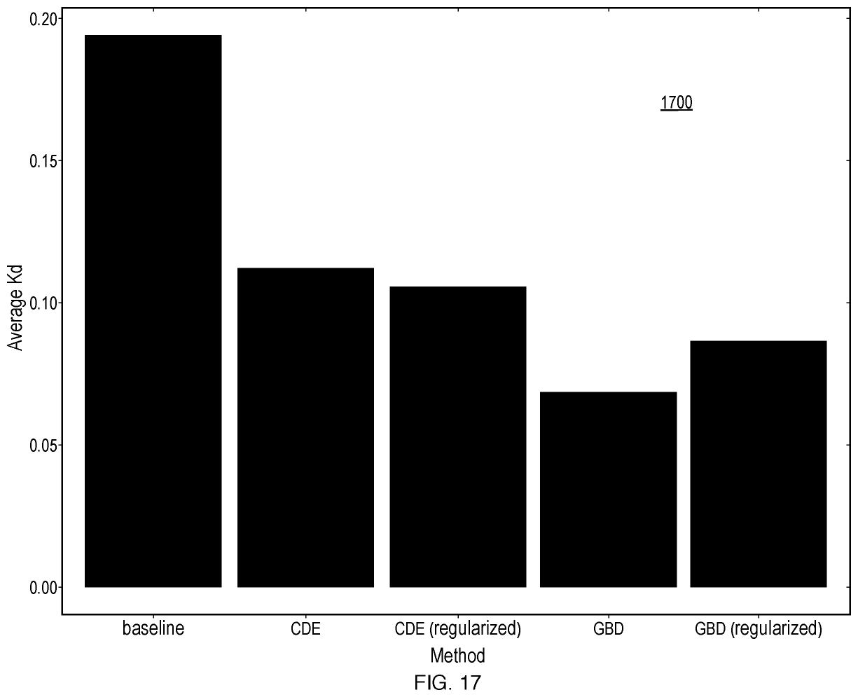

FIG. 17 is a graph 1700 illustrating wet lab data measuring the Kd of the listed protein variants, validating the affinity of the generated proteins.

The methods illustrated by the graph include CDE, regularized and unregularized, GBD, regularized and unregularized, and a baseline process. The dataset that FIG. 17 is based on is illustrated below in Table 4, which lists experimentally measured Kd values for the generated proteins.

TABLE 4

| | | | variant | | |
|---|---|---|---|---|---|
| | variant | variant | parameter | variant | |
| designation | distance | method | group | notes | kd |
| 1 | 2 | CDE | CDE | first order iid CDE | 0.106 |
| | | | (regularized) | | |
| 2 | 2 | CDE | CDE | none iid CDE | 0.112 |
| 3 | 4 | GBD | GBD | first order iid GBD | 0.077 |
| | | | (regularized) | | |
| 4 | 3 | GBD | GBD | first order iid GBD | 0.1 |
| | | | (regularized) | | |
| 5 | 3 | GBD | GBD | first order iid GBD | 0.07 |
| | | | (regularized) | | |
| 6 | 4 | GBD | GBD | first order iid GBD | 0.058 |
| | | | (regularized) | | |
| 7 | 4 | GBD | GBD | first order iid GBD | 0.098 |
| | | | (regularized) | | |
| 8 | 3 | GBD | GBD | first order iid GBD | 0.069 |
| | | | (regularized) | | |
| 9 | 4 | GBD | GBD | first order iid GBD | 0.093 |
| | | | (regularized) | | |
| 10 | 0 | GBD | baseline | baseline | 0.194 |
| 11 | 4 | GBD | GBD | first order iid GBD | 0.07 |
| | | | (regularized) | | |
| 12 | 3 | GBD | GBD | none iid GBD | 0.06 |
| 13 | 3 | GBD | GBD | none iid GBD | 0.085 |
| 14 | 4 | GBD | GBD | none iid GBD | 0.077 |
| 15 | 2 | GBD | GBD | none iid GBD | 0.07 |
| 16 | 4 | GBD | GBD | none iid GBD | 0.066 |
| 17 | 4 | GBD | GBD | none iid GBD | 0.054 |
| 18 | 4 | GBD | GBD | first order medium | 0.141 |
| | | | (regularized) | fitness GBD | |

Wet lab experiments to measure the Kd of our GBD generated variants were conducted as follows. Yeast cells were transformed with clonal plasmids expressing unique anti-FITC scFv designed variants formatted for surface display and including a cMyc tag for expression quantification. After cultivation and scFv expression, yeast cells were stained with the fluorescein antigen as well as a fluorescent conjugated anti-cMyc antibody, at several concentrations. After reaching equilibrium, cells from each concentration stain are measured by flow cytometry. Median fluorescence intensity for fluorescein antigen binding were calculated after gating on expressing cells. Median fluorescence data were fit to a standard single binding affinity curve to determine an approximate binding affinity Kd (dissociation constant) for each clonal scFv variant. These results showed that GBD was superior to other design methods for design FITC antibodies.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The disclosure of the present application also includes the following Illustrative Embodiments:

Illustrative Embodiment 1: A method of engineering an improved biopolymer sequence as assessed by a function, comprising:
(a) providing a starting point in an embedding, optionally wherein the starting point is the embedding of a seed biopolymer sequence, to a system comprising a supervised model that predicts the function of a biopolymer sequence and a decoder network, the supervised model network comprising an encoder network providing the embedding of biopolymer sequences in a functional space representing the function and the decoder network trained to provide a probabilistic biopolymer sequence, given an embedding of a biopolymer sequence in the functional space;
(b) calculating a change in the function with regard to the embedding at the starting point according to a step size, thereby providing a first updated point in the functional space;
(c) optionally calculating a change in the function with regard to the embedding at the first updated point in the functional space and optionally iterating the process of calculating a change in the function with regard to the embedding at a further updated point;
(d) upon approaching a desired level of the function at the first updated point in the functional space, or optionally iterated further updated point, providing the first updated point, or optionally iterated further updated point to the decoder network; and
(e) obtaining a probabilistic improved biopolymer sequence from the decoder.

Illustrative Embodiment 2: A method of engineering an improved biopolymer sequence as assessed by a function, comprising:
(a) providing a starting point in an embedding, optionally wherein the starting point is the embedding of a seed biopolymer sequence, to a system comprising a supervised model network that predicts the function of a biopolymer sequence and a decoder network, the supervised model network comprising an encoder network providing the embedding of biopolymer sequences in a functional space representing the function and the decoder network trained to provide a predicted probabilistic biopolymer sequence, given an embedding of the predicted biopolymer sequence in the functional space;
(b) predicting the function of the starting point in the embedding;
(c) calculating a change in the function with regard to the embedding at the starting point according to a step size, thereby providing a first updated point in the functional space;
(d) providing the first updated point in the functional space to the decoder network to provide a first intermediate probabilistic biopolymer sequence;
(e) providing the first intermediate probabilistic biopolymer sequence to the supervised model to predict the function of the first intermediate probabilistic biopolymer sequence,
(f) calculating the change in the function with regard to the embedding at the first updated point in the functional space to provide a updated point in the functional space;
(g) providing the updated point in the functional space to the decoder network to provide an additional intermediate probabilistic biopolymer sequence;
(h) providing the additional intermediate probabilistic biopolymer sequence to the supervised model to predict the function of the additional intermediate probabilistic biopolymer sequence;
(i) then calculating the change in the function with regard to the embedding at the further first updated point in the functional space to provide a yet further updated point in the functional space, optionally iterating steps (g)-(i), where a yet further updated point in the functional space referenced in step (i) is regarded as the further updated point in the functional space in step (g); and
(j) upon approaching a desired level of the function in the functional space, providing the point in the embedding to the decoder network; and obtaining a probabilistic improved biopolymer sequence from the decoder.

Illustrative Embodiment 3: A non-transient and/or non-transitory computer readable medium comprising instructions that, upon execution by a processor, cause the processor to:
(a) provide a starting point in an embedding, optionally wherein the starting point is the embedding of a seed biopolymer sequence, to a system comprising a supervised model that predicts the function of a biopolymer sequence and a decoder network, the supervised model network comprising an encoder network providing the embedding of biopolymer sequences in a functional space representing the function and the decoder network trained to provide a probabilistic biopolymer sequence, given an embedding of a biopolymer sequence in the functional space;
(b) calculate a change in the function with regard to the embedding at the starting point according to a step size, thereby providing a first updated point in the functional space;
(c) optionally calculate a change in the function with regard to the embedding at the first updated point in the functional space and optionally iterating the process of calculating a change in the function with regard to the embedding at a further updated point;
(d) upon approaching a desired level of the function at the first updated point in the functional space, or optionally iterated further updated point, provide the first updated point, or optionally iterated further updated point to the decoder network; and
(e) obtain a probabilistic improved biopolymer sequence from the decoder.

Illustrative Embodiment 4: A system comprising a processor and non-transient and/or non-transitory computer readable medium comprising instructions that, upon execution by a processor, cause the processor to:
(a) provide a starting point in an embedding, optionally wherein the starting point is the embedding of a seed biopolymer sequence, to a system comprising a supervised model that predicts the function of a biopolymer sequence and a decoder network, the supervised model network comprising an encoder network providing the embedding of biopolymer sequences in a functional space representing the function and the decoder network trained to provide a probabilistic biopolymer sequence, given an embedding of a biopolymer sequence in the functional space;
(b) calculate a change in the function with regard to the embedding at the starting point according to a step size, thereby providing a first updated point in the functional space;
(c) optionally calculate a change in the function with regard to the embedding at the first updated point in the functional space and optionally iterating the process of calculating a change in the function with regard to the embedding at a further updated point;
(d) upon approaching a desired level of the function at the first updated point in the functional space, or optionally iterated further updated point, provide the first updated point, or optionally iterated further updated point to the decoder network; and
(e) obtain a probabilistic improved biopolymer sequence from the decoder.

Illustrative Embodiment 5: A system comprising a processor and non-transient and/or non-transitory computer readable medium comprising instructions that, upon execution by a processor, cause the processor to:
(a) provide a starting point in an embedding, optionally wherein the starting point is the embedding of a seed biopolymer sequence, to a system comprising a supervised model network that predicts the function of a biopolymer sequence and a decoder network, the supervised model network comprising an encoder network providing the embedding of biopolymer sequences in a functional space representing the function and the decoder network trained to provide a predicted probabilistic biopolymer sequence, given an embedding of the predicted biopolymer sequence in the functional space;
(b) predict the function of the starting point in the embedding;
(c) calculate a change in the function with regard to the embedding at the starting point according to a step size, thereby providing a first updated point in the functional space;
(d) provide the first updated point in the functional space to the decoder network to provide a first intermediate probabilistic biopolymer sequence;
(e) provide the first intermediate probabilistic biopolymer sequence to the supervised model to predict the function of the first intermediate probabilistic biopolymer sequence,
(f) calculate the change in the function with regard to the embedding at the first updated point in the functional space to provide a updated point in the functional space;
(g) provide the updated point in the functional space to the decoder network to provide an additional intermediate probabilistic biopolymer sequence;
(h) provide the additional intermediate probabilistic biopolymer sequence to the supervised model to predict the function of the additional intermediate probabilistic biopolymer sequence;
(i) then calculate the change in the function with regard to the embedding at the further first updated point in the functional space to provide a yet further updated point in the functional space, optionally iterating steps (g)-(i), where a yet further updated point in the functional space referenced in step (i) is regarded as the further updated point in the functional space in step (g); and
(j) upon approaching a desired level of the function in the functional space, provide the point in the embedding to the decoder network; and obtaining a probabilistic improved biopolymer sequence from the decoder.

Illustrative Embodiment 6: A non-transient and/or non-transitory computer readable medium comprising instructions that, upon execution by a processor, cause the processor to:
(a) provide a starting point in an embedding, optionally wherein the starting point is the embedding of a seed biopolymer sequence, to a system comprising a supervised model network that predicts the function of a biopolymer sequence and a decoder network, the supervised model network comprising an encoder network providing the embedding of biopolymer sequences in a functional space representing the function and the decoder network trained to provide a predicted probabilistic biopolymer sequence, given an embedding of the predicted biopolymer sequence in the functional space;
(b) predict the function of the starting point in the embedding;
(c) calculate a change in the function with regard to the embedding at the starting point according to a step size, thereby providing a first updated point in the functional space;
(d) provide the first updated point in the functional space to the decoder network to provide a first intermediate probabilistic biopolymer sequence;
(e) provide the first intermediate probabilistic biopolymer sequence to the supervised model to predict the function of the first intermediate probabilistic biopolymer sequence,
(f) calculate the change in the function with regard to the embedding at the first updated point in the functional space to provide a updated point in the functional space;
(g) provide the updated point in the functional space to the decoder network to provide an additional intermediate probabilistic biopolymer sequence;
(h) provide the additional intermediate probabilistic biopolymer sequence to the supervised model to predict the function of the additional intermediate probabilistic biopolymer sequence;
(i) then calculate the change in the function with regard to the embedding at the further first updated point in the functional space to provide a yet further updated point in the functional space, optionally iterating steps (g)-(i), where a yet further updated point in the functional space referenced in step (i) is regarded as the further updated point in the functional space in step (g); and
(j) upon approaching a desired level of the function in the functional space, provide the point in the embedding to the decoder network; and obtaining a probabilistic improved biopolymer sequence from the decoder.

1. A method of engineering an improved biopolymer sequence as assessed by a function, comprising:
(a) providing a starting point in an embedding to a system comprising a supervised model that predicts the function of a biopolymer sequence and a decoder network, the supervised model network comprising an encoder network providing the embedding of biopolymer sequences in a functional space representing the function, and the decoder network trained to provide a probabilistic biopolymer sequence, given an embedding of a biopolymer sequence in the functional space;
(b) calculating a change in the function in relation to the embedding at the starting point according to a step size, the calculated change enabling providing a first updated point in the functional space;
(c) upon reaching a desired level of the function within a particular threshold at the first updated point in the functional space providing the first updated point; and
(d) obtaining a probabilistic improved biopolymer sequence from the decoder.

2. The method of claim 1, wherein the starting point is the embedding of a seed biopolymer sequence.

3. The method of claim 1, further comprising:
calculating a second change in the function with regard to the embedding at the first updated point in the functional space; and
iterating the process of calculating the second change in the function with regard to the embedding at a further updated point.

4. The method of claim 3, wherein providing the first updated point can be performed upon reaching a desired level of the function within a particular threshold at the optionally iterated further updated point, and providing the further updated point includes providing the iterated further updated point to the decoder network.

5. The method of claim 1, wherein the embedding is a continuously differentiable functional space representing the function and having one or more gradients.

6. The method of claim 1, wherein calculating the change of the function with regard to the embedding comprises taking a derivative of the function with regard to the embedding.

7. The method of claim 1, wherein the function is a composite function of two or more component functions.

8. The method of claim 7, wherein the composite function is a weighted sum of the two or more composite functions.

9. The method of claim 1, wherein two or more starting points in the embedding are used concurrently.

10. The method of claim 1, wherein correlations between residues in a probabilistic sequence comprising a probability distribution of residue identities are considered in a sampling process using conditional probabilities that account for the portion of the sequence that has already been generated.

11. The method of claim 1, further comprising selecting the maximum likelihood improved biopolymer sequence from a probabilistic biopolymer sequence comprising a probability distribution of residue identities.

12. The method of claim 1, comprising sampling the marginal distribution at each residue of a probabilistic biopolymer sequence comprising a probability distribution of residue identities.

13. The method of claim 1, wherein the change of the function with regard to the embedding, is calculated by calculating the change of the function with regard to the encoder, then the change of the encoder to the change of the decoder, and the change of the decoder with regard to the embedding.

14. The method of claim 1, the method comprising:
providing the first updated point in the functional space or further updated point in the functional space to the decoder network to provide an intermediate probabilistic biopolymer sequence,
providing the intermediate probabilistic biopolymer sequence to the supervised model network to predict the function of the intermediate probabilistic biopolymer sequence,
calculating the change in the function with regard to the embedding for the intermediate probabilistic biopolymer to provide a further updated point in the functional space.

15-16. (canceled)

17. The method of claim 1, wherein the biopolymer is a protein.

18-19. (canceled)

20. The method of claim 1, wherein the encoder is trained using a training data set of at least 20 biopolymer sequences.

21-87. (canceled)

88. A system comprising a processor and non-transitory computer readable medium comprising instructions that, upon execution by a processor, cause the processor to:
(a) predict the function of a starting point in an embedding at a to a system comprising a supervised model network that predicts the function of a biopolymer sequence and a decoder network, the supervised model network comprising an encoder network providing the embedding of biopolymer sequences in a functional space representing the function and the decoder network trained to provide a predicted probabilistic biopolymer sequence, given an embedding of the predicted biopolymer sequence in the functional space;
(b) calculate a change in the function in relation to the embedding at the starting point according to a step size, thereby enabling providing a first updated point in the functional space;
(c) calculate, at the decoder network, a first intermediate probabilistic biopolymer sequence based on the first updated point in the functional space;
(d) predict the function of the first intermediate probabilistic biopolymer sequence, at the supervised model based on the first intermediate biopolymer sequence;
(e) calculate the change in the function with regard to the embedding at the first updated point in the functional space to provide an updated point in the functional space;
(f) calculate an additional intermediate probabilistic biopolymer sequence at the decoder network based on the updated point in the functional space;
(g) predict the function of the additional intermediate probabilistic biopolymer sequence, at the supervised model, based on the additional intermediate probabilistic biopolymer sequence;
(h) calculate the change in the function with regard to the embedding at the further first updated point in the functional space to provide a yet further updated point in the functional space, optionally iterating steps (g)-(i), where a yet further updated point in the functional space referenced in step (i) is regarded as the further updated point in the functional space in step (g); and
(i) upon approaching a desired level of the function in the functional space, provide the point in the embedding to the decoder network; and obtaining a probabilistic improved biopolymer sequence from the decoder.

89. (canceled)

90. A method of making a biopolymer comprising synthesizing an improved biopolymer sequence obtainable by the method of claim 1.

91-117. (canceled)

118. A method for training a supervised model for use in the method of claim 1, wherein this supervised model comprises an encoder network that is configured to map biopolymer sequences to representations in an embedding functional space, wherein the supervised model is configured to predict a function of the biopolymer sequence based on the representations, and wherein the method comprises the steps of:
(a) providing a plurality of training biopolymer sequences, wherein each training biopolymer sequence is labelled with a function;
(b) mapping, using the encoder, each training biopolymer sequence to a representation in the embedding functional space;
(c) predicting, using the supervised model, based on these representations, the function of each training biopolymer sequence;
(d) determining, using a predetermined prediction loss function, for each training biopolymer sequence, how well the predicted function is in agreement with the function as per the label of the respective training biopolymer sequence; and
(e) optimizing parameters that characterize the behavior of the supervised model with the goal of improving the rating by said prediction loss function that results when further training biopolymer sequences are processed by the supervised model.

119. A method for training a decoder for use in a method or system according to claim 1, wherein the decoder is configured to map a representation of a biopolymer sequence from an embedding functional space to a probabilistic biopolymer sequence, comprising the steps of:
(a) providing a plurality of representations of biopolymer sequences in the embedding functional space;
(b) mapping, using the decoder, each representation to a probabilistic biopolymer sequence;
(c) drawing a sample biopolymer sequence from each probabilistic biopolymer sequence;
(d) mapping, using a trained encoder, this sample biopolymer sequence to a representation in said embedding functional space;
(e) determining, using a predetermined reconstruction loss function, how well each so-determined representation is in agreement with the corresponding original representation; and
(f) optimizing parameters that characterize the behavior of the decoder with the goal of improving the rating by said reconstruction loss function that results when further representations of biopolymer sequences from said embedding functional space are processed by the decoder.

120. (canceled)

121. A method for training an ensemble of a supervised model and a decoder,
wherein the supervised model comprises an encoder network that is configured to map biopolymer sequences to representations in an embedding functional space,
wherein the supervised model is configured to predict a function of the biopolymer sequence based on the representations,
wherein the decoder is configured to map a representation of a biopolymer sequence from an embedding functional space to a probabilistic biopolymer sequence,
and wherein the method comprises the steps of:
(a) providing a plurality of training biopolymer sequences, wherein each training biopolymer sequence is labelled with a function;
(b) mapping, using the encoder, each training biopolymer sequence to a representation in the embedding functional space;
(c) predicting, using the supervised model, based on these representations, the function of each training biopolymer sequence;
(d) mapping, using the decoder, each representation in the embedding functional space to a probabilistic biopolymer sequence;
(e) drawing a sample biopolymer sequence from the probabilistic biopolymer sequence;
(f) determining, using a predetermined prediction loss function, for each training biopolymer sequence, how well the predicted function is in agreement with the function as per the label of the respective training biopolymer sequence;
(g) determining, using a predetermined reconstruction loss function, for each sample biopolymer sequence, how well it is in agreement with the original training biopolymer sequence from which it was produced;
(h) optimizing parameters that characterize the behavior of the supervised model and parameters that characterize the behavior of the decoder with the goal of improving the rating by a predetermined combination of the prediction loss function and the reconstruction loss function.

122. A set of parameters that characterize the behavior of a supervised model, an encoder or a decoder, obtained by the method of claim 118.
